(19) United States
(12) Patent Application Publication
(10) Pub. No.: US 2025/0082922 A1
(43) Pub. Date: Mar. 13, 2025

(54) GUIDEWIRE

(71) Applicant: Kardion GmbH, Stuttgart (DE)

(72) Inventors: Attila Fabiunke, Karlsruhe (DE); Leon Wenning, Stuttgart (DE); Hardy Baumbach, Stuttgart (DE)

(21) Appl. No.: 18/580,919

(22) PCT Filed: Jul. 20, 2022
(86) PCT No.: PCT/US2022/037685

§ 371 Date: Jan. 19, 2024

Related U.S. Application Data
(60) Provisional application No. 63/224,326, filed on Jul. 21, 2021.

(51) Int. Cl. A61M 60/867; A61M 25/09; A61M 60/165

(52) U.S. Cl. CPC A61M 60/867; A61M 25/09; A61M 60/165; A61M 2025/09083; A61M 2205/0216; A61M 2205/32; A61M 2205/583; A61M 2210/125; A61M 2210/127

(57) ABSTRACT

A guidewire configured to traverse through a catheter with-out exiting side holes of the catheter for placement of a minimally invasive miniaturized percutaneous mechanical circulatory support device or ventricular assist device across the heart. The guidewire includes a proximal end, a distal end, and an elongate flexible body extending therebetween. The guidewire can have a variable flexibility across its length via variable diameters and tapered sections that make up its core, and one or more coils surrounding and connected to sections of its core to prevent kinking. The guidewire can have one or more radiopaque markers and one or more visual markers to facilitate use.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Application No. 63/224,326, titled GUIDEWIRE and filed on Jul. 21, 2021, the entire contents of which is incorporated by reference herein in its entirety for all purposes and forms a part of this specification.

### BACKGROUND

Mechanical circulatory support systems may be used to assist with pumping blood during various medical procedures and/or as therapy for certain cardiac conditions. For example, cardiogenic shock (CS) is a common cause of mortality, and management remains challenging despite advances in therapeutic options. CS is caused by severe impairment of myocardial performance that results in diminished cardiac output. end-organ hypoperfusion, and hypoxia. Clinically this presents as hypotension refractory to volume resuscitation with features of end-organ hypoperfusion requiring immediate pharmacological or mechanical intervention. Acute myocardial infarction (MI) accounts for over about 80% of patients in CS.

Percutaneous coronary intervention (PCI) is a non-surgical procedure to revascularize stenotic coronary arteries. PCI includes a variety of techniques, e.g. balloon angioplasty, stent implantation, rotablation and lithotripsy. A PCI is considered high risk if either the patient has relevant comorbidities (e.g. frailty or advanced age), the PCI per se is very complex (e.g. bifurcation or total occlusions) or hemodynamic status is challenging (e.g. impaired ventricular function).

Miniature, catheter-based intracardiac blood pumps have been developed for percutaneous insertion into a patient's body as an acute therapy for CS and for temporary assistance during PCI. However, existing solutions for mechanical circulatory support systems have various performance deficiencies such as, for example, inadequate blood flow, the requirement for ongoing motor purging within the pump, undesirably high hemolysis, and inadequate sensing of hemodynamic parameters. Furthermore, existing mechanical circulatory support systems can be difficult to place in the desired location in the body, difficult to maintain in position during a procedure, difficult to visualize, and difficult to use. Thus, there remains a need for mechanical circulatory support systems with features that overcome these and other drawbacks.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for mechanical circulatory support systems.

The following description describes non-limiting examples of some embodiments of mechanical circulatory support devices, systems, and methods. Other embodiments of the disclosed systems and methods may or may not include the features described herein. Moreover, disclosed advantages and benefits can apply only to certain embodiments and should not be used to limit the disclosure.

A first aspect relates to a guidewire configured to traverse a catheter having one or more side holes. The guidewire includes a proximal end, a distal end, the distal end having a distal advance segment configured to traverse distally through the catheter without exiting the side holes of the catheter, and an elongate flexible body extending between the proximal end and the distal end, the elongate flexible body having a distal region extending between a distal transition and the distal end, the distal region having a spiral coil geometry.

A second aspect relates to the guidewire of the first aspect, wherein the distal advance segment includes an elongate straight tip.

A third aspect relates to the guidewire of the first or second aspects, wherein the distal advance segment has a length greater than a maximum diameter of the one or more side holes.

A fourth aspect relates to the guidewire of any of the preceding aspects, wherein a minimum length of the distal advance segment is between about 0.5 mm and about 3.5 mm.

A fifth aspect relates to the guidewire of any of the preceding aspects, wherein a maximum angle between a central axis of the distal advance segment and a longitudinal axis of the catheter is between about 17° and about 25°.

A sixth aspect relates to the guidewire of the first aspect, wherein the distal advance segment has a curved advance segment extending from an inflection point at a distal end of the spiral coil configuration.

A seventh aspect relates to the guidewire of the sixth aspect, wherein the spiral coil configuration is concave in a first direction and the distal advance segment is concave in a second direction.

An eighth aspect relates to the guidewire of the sixth aspect, wherein the distal advance segment includes a first curved region with a first inflection point and a second curved region with a second inflection point.

A ninth aspect relates to the guidewire of the eighth aspect, wherein the first curved region and/or the second curved region includes an arc length greater than a maximum diameter of the one or more side holes.

A tenth aspect relates to the guidewire of the ninth aspect, wherein the arc length is greater than 0.5 mm.

An eleventh aspect relates to the guidewire of any of the eighth to tenth aspects, wherein a maximum angle between a longitudinal axis of the catheter and a central axis of a portion of the advance segment extending distally from the second inflection point is between about 5° and about 85°.

A twelfth aspect relates to the guidewire of the eleventh aspect, wherein the maximum angle between the longitudinal axis of the catheter and the central axis of the portion of the advance segment extending distally from the second inflection point is between about 10° and about 60°.

A thirteenth aspect relates to the guidewire of any of the eight to twelfth aspects, wherein a maximum length of a portion of the advance segment extending distally from the second inflection point is between about 0.3 mm and about 4 mm.

A fourteenth aspect relates to the guidewire of any of the eighth to thirteenth aspects, wherein a radius of curvature of the first inflection point and/or the second inflection point is greater than a radius of the one or more side holes.

A fifteenth aspect relates to the guidewire of the fourteenth aspect, wherein the radius of curvature of the first inflection point and/or the second inflection point is between about 0.5 mm and about 0.8 mm.

A sixteenth aspect relates to the guidewire of any of the preceding aspects, wherein a diameter at a largest cross-section of the advance segment is greater than or equal to a diameter of the one or more side holes.

A seventeenth aspect relates to the guidewire of any of the preceding aspects, wherein the advance segment is spheroidal in shape.

An eighteenth aspect relates to the guidewire of any of the preceding aspects, wherein the diameter at the largest cross-section of the advance segment is between 0.8 mm and 1 mm.

A nineteenth aspect relates to the guidewire of any of the preceding aspects, wherein the distal end is rounded in shape.

A twentieth aspect relates to the guidewire of any of the preceding aspects, further including a proximal region extending between a proximal transition and the proximal end, wherein the proximal region is configured to facilitate movement of the guidewire through a non-linear path.

A twenty-first aspect relates to a method of delivering a device to a cardiovascular system of a patient. The method includes delivering a first guidewire to the cardiovascular system of the patient, advancing a catheter over the first guidewire, the catheter having one or more side holes, removing the first guidewire from the catheter, and advancing a second guidewire through the catheter, wherein the second guidewire is the guidewire of any of the preceding aspects and is configured to bypass the holes of the catheter as the second guidewire advances through the catheter. The method also includes removing the catheter from the second guidewire, feeding a proximal end of the second guidewire into a distal end of the device, and advancing the device over the second guidewire into the cardiovascular system of the patient.

A twenty-second aspect relates to the method of the twenty-first aspect, wherein the device includes a heart pump.

A twenty-third aspect relates to the method of either the twenty-first aspect or the twenty-second aspect, wherein the first guidewire has a diameter of 0.035 inches.

A twenty-fourth aspect relates to the method of any of the twenty-first to twenty-third aspects, wherein the second guidewire has a diameter of 0.018 inches.

Disclosed herein is a guidewire configured to traverse a catheter having one or more side holes, the guidewire comprising a proximal end, a distal end, and an elongate flexible body extending between the proximal end and the distal end. The distal end can comprise a distal advance segment configured to traverse distally through the catheter without exiting the side holes of the catheter. The elongate flexible body can comprise a distal region extending between a distal transition and the distal end, the distal region comprising a spiral coil geometry.

In the above guidewire or in other implementations as described herein, one or more of the following features can also be provided. In some implementations, the distal advance segment comprises an elongate straight tip. In some implementations, the distal advance segment comprises a length greater than a maximum diameter of the one or more side holes. In some implementations, a minimum length of the distal advance segment is between about 0.5 mm and about 3.5 mm. In some implementations, a maximum angle between a central axis of the distal advance segment and a longitudinal axis of the catheter is between about 17° and about 25°. In some implementations, the distal advance segment comprises a curved advance segment extending from an inflection point at a distal end of the spiral coil geometry. In some implementations, the spiral coil geometry is concave in a first direction and the distal advance segment is concave in a second direction. In some implementations, the distal advance segment comprises a first curved region with a first inflection point and a second curved region with a second inflection point. In some implementations, the first curved region and/or the second curved region comprises an arc length greater than a maximum diameter of the one or more side holes. In some implementations, the arc length is greater than about 0.5 mm. In some implementations, a maximum angle between a longitudinal axis of the catheter and a central axis of a portion of the distal advance segment extending distally from the second inflection point is between about 5° and about 85°. In some implementations, the maximum angle between the longitudinal axis of the catheter and the central axis of the portion of the distal advance segment extending distally from the second inflection point is between about 10° and about 60°. In some implementations, a maximum length of a portion of the distal advance segment extending distally from the second inflection point is between about 0.3 mm and about 4 mm. In some implementations, a radius of curvature of the first inflection point and/or the second inflection point is greater than a radius of the one or more side holes. In some implementations, the radius of curvature of the first inflection point and/or the second inflection point is between about 0.5 mm and about 0.8 mm. In some implementations, a diameter at a largest cross-section of the distal advance segment is greater than or equal to a diameter of the one or more side holes. In some implementations, the distal advance segment is spheroidal in shape. In some implementations, a diameter at a largest cross-section of the distal advance segment is between about 0.8 mm and about 1 mm. In some implementations, the distal end is rounded in shape. In some implementations, the guidewire further comprises a proximal region extending between a proximal transition and the proximal end, wherein the proximal region is configured to facilitate movement of the guidewire through a non-linear path. In some implementations, the elongate flexible body comprises a core comprising a plurality of segments having different diameters. In some implementations, the diameters of each of the plurality of segments of the core are between about 0.10 mm and about 0.5 mm. In some implementations, the plurality of segments having different diameters are connected to one another by one or more tapered, chamfered, conical. or frustoconical transition segments, each of the tapered, chamfered, conical, or frustoconical transition segments having a varying diameter across its length. In some implementations, the guidewire has a variable flexibility along its length. In some implementations, the elongate flexible body further comprises one or more coils of wire surrounding the core. In some implementations, the one or more coils of wire surrounding the core prevent the guidewire from kinking. In some implementations, the one or more coils of wire surround at least a portion of the core of the distal region and/or the proximal region. In some implementations, the elongate flexible body further comprises a body region and/or a pump region. In some implementations, the distal region and/or the proximal region have a diameter smaller than a diameter of the body region and/or the pump region. In some implementations, the one or more coils of wire surround at least a portion of the core of the body region and/or the pump region. In some implementations, the guidewire has a length of between about 2770 mm and 4030 mm. In some implementations, the distal region of the guidewire comprises one or more radiopaque markers. In some implementations, the distal region of the guidewire comprises a first radiopaque marker at or adjacent the distal end, a second radiopaque marker between about 20 mm and about 180 mm from the distal end, and/or a third radiopaque marker about 200 mm from the distal end. In some implementations, the guidewire comprises a plurality of radiopaque markers spaced evenly along at least a portion of its length to provide a scale that can be visualized by fluoroscopy. In some implementations, the proximal region of the guidewire comprises one or more visual markers.

Disclosed herein is a method of delivering a device to a cardiovascular system of a patient, the method comprising: delivering a first guidewire to the cardiovascular system of the patient; advancing a catheter over the first guidewire, the catheter comprising one or more side holes, removing the first guidewire from the catheter; advancing a second guidewire through the catheter, wherein the second guidewire comprises the guidewire of any one of Claims 1-35, wherein the second guidewire is configured to bypass the one or more side holes of the catheter as the second guidewire advances through the catheter; removing the catheter from the second guidewire; feeding a proximal end of the second guidewire into a distal end of the device; and advancing the device over the second guidewire into the cardiovascular system of the patient.

In the above method or in other implementations as described herein, one or more of the following features can also be provided. In some implementations, the device comprises a heart pump. In some implementations, the first guidewire has an outer diameter of about 0.035 inches. In some implementations, the second guidewire has a minimum outer diameter of about 0.018 inches. In some implementations, advancing the second guidewire into the cardiovascular system of the patient comprises advancing the second guidewire so that the first radiopaque marker and/or the second radiopaque marker are positioned within a left ventricle of the patient and the third radiopaque marker is positioned within an aorta of the patient.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1A is a cross sectional rendering of a guidewire positioned across an aortic valve and extending into a left ventricle of a heart according to some embodiments.

FIG. 1B is a cross sectional rendering of a guidewire positioning a mechanical circulatory support (MCS) device carried by a catheter across an aortic valve according to some embodiments.

FIG. 2 schematically illustrates an MCS system inserted into the body over a guidewire via the access pathway from the femoral artery to the left ventricle according to some embodiments.

FIG. 3 is a side elevational view of a guidewire inserted through an MCS system according to some embodiments.

FIG. 4 shows an embodiment of a placement guidewire.

FIGS. 5A-5C show embodiments of a distal portion of a placement guidewire.

FIG. 6A shows an embodiment of a distal portion of a placement guidewire having an elongated distal advance segment.

FIG. 6B shows an enlarged view of the distal advance segment of the guidewire shown in FIG. 6A.

FIG. 6C shows an enlarged cross-sectional view of the distal advance segment of the guidewire of FIG. 6A positioned within a catheter.

FIG. 7A shows an embodiment of a distal portion of a placement guidewire having a curved distal advance segment.

FIG. 7B shows an enlarged view of the distal advance segment of the guidewire shown in FIG. 7A.

FIG. 7C shows an embodiment of a distal portion of a placement guidewire having a curved distal advance segment.

FIG. 7D shows an enlarged cross-sectional view of the distal advance segment of the guidewire of FIG. 7A positioned within a catheter.

FIG. 8A shows an embodiment of a distal portion of a placement guidewire having an enlarged distal advance segment.

FIG. 8B shows an enlarged cross-sectional view of a guidewire having an enlarged distal advance segment positioned within a catheter.

FIG. 9A shows a cross-sectional view of a portion of an embodiment of a distal region of a guidewire

FIG. 9B shows a cross-sectional view of a portion of an embodiment of a distal region of a guidewire.

FIG. 10A shows an embodiment of a guidewire.

FIG. 10B shows a cross-sectional view of the guidewire of FIG. 10A.

FIG. 11A shows an embodiment of a guidewire.

FIG. 11B shows a cross-sectional view of the guidewire of FIG. 11A.

FIG. 11C shows a sectional view of the cross-section of the guidewire of FIG. 11B.

FIG. 12 shows a proximal region of a guidewire according to some embodiments.

FIG. 13 shows a proximal region of a guidewire according to some embodiments.

FIG. 14 shows a proximal region of a guidewire according to some embodiments.

FIG. 15 shows a perspective view of a distal, pump region of an MCS device according to some embodiments.

FIG. 16 show a side elevational view of a distal region of the MCS device of FIG. 15, showing a removable guidewire aid with a guidewire guide tube defining a guidewire path in place.

FIG. 17 shows an enlarged cross-sectional view of a portion of the distal region of the MCS device showing a portion of the guidewire path.

FIG. 18 shows a perspective view of a bend relief of the MCS device.

FIG. 19 shows a cross-sectional view of a portion of the distal region of the MCS device showing a portion of the guidewire path.

FIGS. 20A-C show top, side, and side views of a guidewire port 80 within the bend relief.

FIG. 21 shows a perspective view of a portion of the distal region of the MCS device showing a connection between a motor housing and the bend relief.

While the above-identified drawings set forth presently disclosed embodiments, other embodiments are also contemplated, as noted in the detailed description. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

Guidewires are provided for use in minimally invasive procedures. Certain embodiments of guidewires described herein can be used in mechanical circulatory support systems or mechanical left ventricular support systems, for example, for the placement of mechanical circulatory support (MCS) devices or ventricular assist devices (VADs). Guidewires described herein can advantageously improve ease of use and/or address challenges associated with use of MCS devices or VADs. For example, guidewires described herein can improve the ability to detect guidewire migration without needing to inject contrast to visualize patient anatomy, improve the ability to decide how deep in the left ventricle of a patient to place the MCS device/VAD, improve the ability to visualize relative scale, improve guidewire handling, control, and/or pushability, and/or allow for a faster procedure.

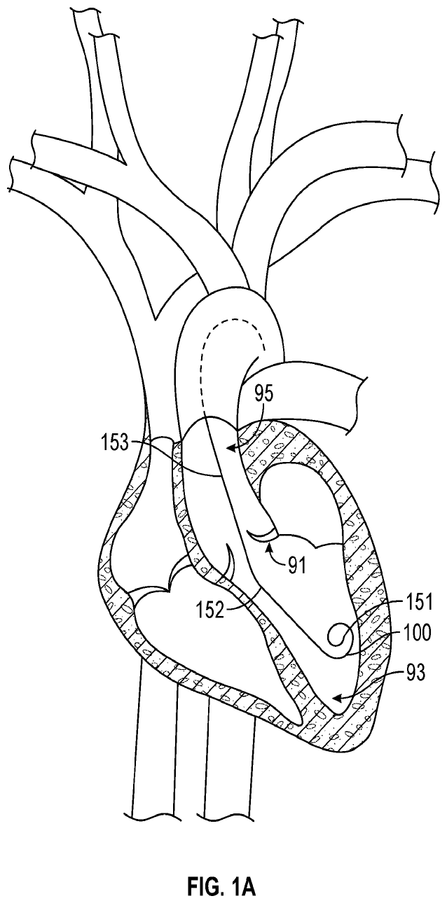

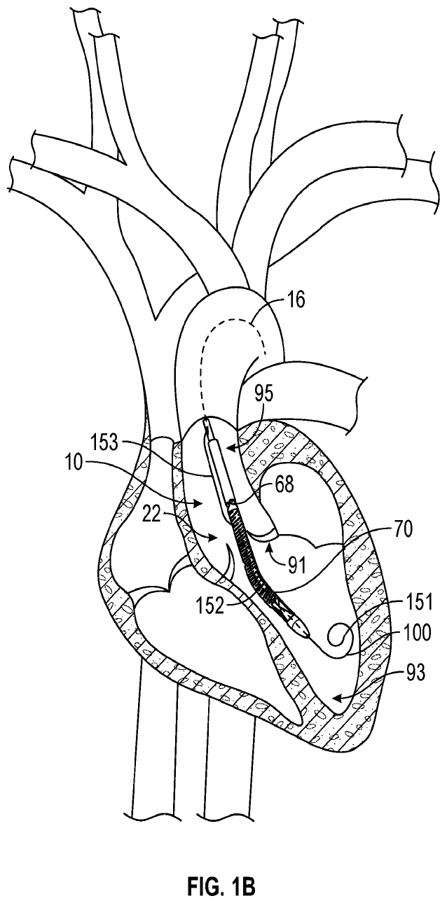

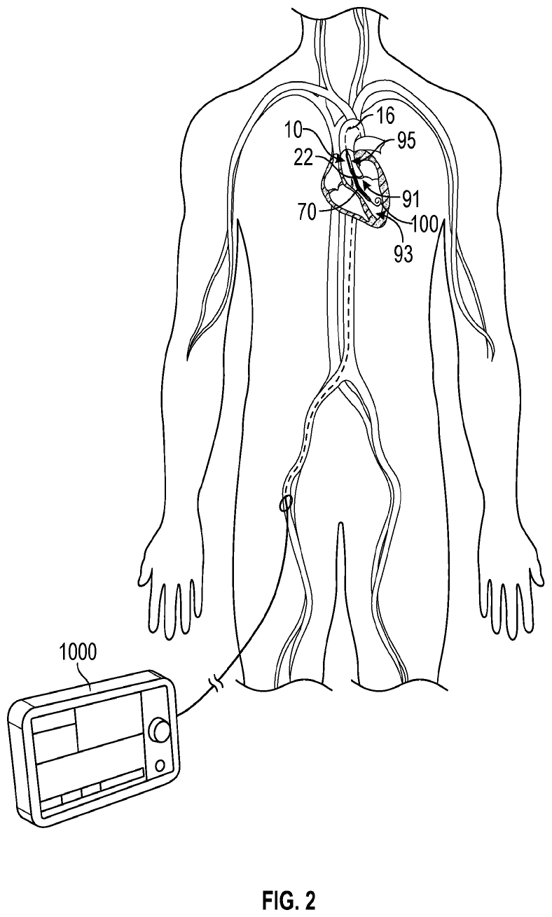

FIG. 1A is a schematic of a guidewire 100 positioned across an aortic valve 91 with its distal end positioned in a left ventricle 93. FIG. 1B is a schematic of a distal end of an embodiment of an MCS system 10 having a pump 22 mounted on the end of a catheter 16 placed in the heart over the guidewire 100. FIG. 2 schematically illustrates an MCS system 10 inserted into the body via an access pathway from the femoral artery to the left ventricle according to some embodiments over the guidewire 100. Access may be accomplished via a transfemoral, a transaxillary, a transaortal, a transapical approach, or the like.

As shown in FIG. 1A, a distal region of the guidewire 100 can include one or more radiopaque markers to aid in positioning the guidewire 100 within the cardiovascular system of a patient and/or to ensure it, as well as any device such as an MCS device or VAD positioned by the guidewire 100, stays in the desired position. For example, the guidewire 100 can include a first radiopaque marker 151 at or adjacent the distal end of the guidewire 100. In some embodiments, the guidewire 100 can include a second radiopaque marker 152 positioned proximally from the distal end. For example, the second radiopaque marker 152 can be positioned between 20 mm or about 20 mm and 180 mm or about 180 mm from the distal end. In certain embodiments, the guidewire 100 can include a third radiopaque marker 153 which can be positioned proximally from the distal end. The third radiopaque marker 153 can be positioned 200 mm or about 200 mm from the distal end. The guidewire 100 may include any of the first radiopaque marker 151, the second radiopaque marker 152, and the third radiopaque marker 153 alone or in combination with either or both of the other radiopaque markers.

In certain embodiments, when correctly positioned within the cardiovascular system, the first radiopaque marker 151 and/or the second radiopaque marker 152 can be positioned within the left ventricle 93 and the third radiopaque marker can be positioned within the ascending aorta 95. In some embodiments, the second radiopaque marker 152 is located between 100 mm or about 100 mm to 180 mm or about 180 mm, preferably 160 mm or about 160 mm, or located between 20 mm or about 20 mm to 60 mm or about 60 mm, preferably 40 mm or about 40 mm, from the first radiopaque marker 151. The guidewire 100 may be positioned such that the aortic valve 91 is located between the third radiopaque marker 153 and the first or second radiopaque markers 151/152. In some embodiments, the guidewire 100 comprises a plurality of radiopaque markers along at least a portion of its length to provide a scale that can be visualized by fluoroscopy. Such plurality of radiopaque markers can be disposed along or adjacent a distal region of the guidewire 100. Furthermore, such plurality of radiopaque markers can be evenly spaced or substantially evenly spaced, for example, one every 10 mm or about every 10 mm, along at least a portion of the guidewire 100. Such plurality of radiopaque markers can be used by a physician/user as a guide to decide how deep to place an MCS device and/or VAD, and/or used to capture information that can be used for subsequent treatment or therapies (e.g., such as measurements of patient-specific anatomy). When included in the guidewire 100, a radiopaque marker can be used as a guide for positioning an MCS device and/or VAD, particularly if the MCS device and/or VAD has its own radiopaque marker (e.g., relative positions of a radiopaque marker of the guidewire 100 and a radiopaque marker of the MCS device and/or VAD can be used to ensure proper positioning of the MCS device and/or VAD to the guidewire and/or the patient anatomy).

As shown in FIG. 1B, in some embodiments, the MCS system 10 may include a low-profile axial rotary blood pump 22 mounted on the catheter 16, such as an 8 French (Fr) catheter or a catheter no larger than about 10.5 Fr. In some embodiments, an inlet tube 70 of the pump 22 extends across the aortic valve 91. An impeller may be located at the outflow section 68 (also referred to as a pump outlet herein) of the inlet tube 70, drawing blood from the left ventricle 93 through the inlet tube 70 and ejecting it out the outflow section 68 into the ascending aorta 95. The motor may be mounted directly proximal to the impeller in a sealed housing, eliminating the need to purge or flush the motor prior to or during use. This configuration provides hemodynamic support during high-risk PCI, with sufficient time and safety for a complete revascularization via a minimally invasive approach (rather than an open surgical procedure). The MCS system 10 or portions thereof may be visualized fluoroscopically, eliminating the need for placement using sensors. Also shown in FIG. 1B are the first radiopaque marker 151, the second radiopaque marker 152, and the third radiopaque marker 153 of the guidewire 100 after the MCS system 10 has been positioned in the desired location over the guidewire 100. The position of the guidewire 100 within an interior of the inlet tube 70 is indicated by a dashed line in FIG. 1B.

In some embodiments, the MCS system 10 actively unloads the left ventricle by pumping blood from the ventricle into the ascending aorta and systemic circulation. When in place, the MCS device may be driven by a complementary MCS Controller 1000 to provide between 0.4 l/min up to 6.0 l/min of partial left ventricular support at about 60 mmHg pressure differential. In some embodiments, the MCS system 10 may include a 14 Fr to an 18 Fr, or a 13 Fr to a 19 Fr, axial rotary blood pump and inlet tube assembly mounted on the catheter 16.

In general, the overall MCS system 10 may include a series of related subsystems and accessories, including one or more of the following: The MCS system 10 may include a pump, shaft, proximal hub, insertion tool, proximal cable, infection shield, guidewire guide tube and/or guidewire aid. The pump 22 may be provided sterile. An MCS catheter 16 may contain the electrical cables and a guidewire lumen for over-the-wire insertion. The proximal hub can contain a guidewire outlet with a valve to maintain hemostasis and connects the MCS catheter 16 to the proximal cable, that connects the pump 22 to the controller 1000. The proximal cable 28 may be 3.5 m (approx. 177 inch) or about 3.5 m in length and extend from a sterile field to a non-sterile field where the controller 1000 is located. An MCS insertion tool may be provided pre-mounted on the MCS device to facilitate the insertion of the pump into the introducer sheath and to protect the inlet tube and the valves from potential damage or interference when passing through the introducer sheath. A peel-away guidewire aid may be pre-mounted on the MCS device to facilitate the insertion of a placement guidewire, such as guidewire as described herein, into the pump 22 and into the MCS catheter 16, optionally with the MCS insertion tool also pre-mounted such that the guidewire guide tube may pass at least in part through a space between the MCS device and the MCS insertion tool. A 3 m or about 3 m in length, 0.018″ in diameter placement guidewire having a soft coiled pre-shaped tip for atraumatic wire placement into the left ventricle or any of the guidewires as described herein may be used. The guidewire may be provided sterile. A 14 Fr or 16 Fr introducer sheath may be used with a usable length of 275 mm to maintain access into the femoral artery and provide hemostasis for a first guidewire (e.g., 0.035″ in diameter), a diagnostic catheter, the 0.018″ placement guidewire or any of the guidewires as described herein, and the insertion tool. The housing of the introducer sheath may be designed to accommodate the MCS insertion tool. The introducer sheath can be provided sterile. An introducer dilator may be compatible with the introducer sheath to facilitate atraumatic insertion of the introducer sheath into the femoral artery. The introducer dilator can be provided sterile. The controller 1000 may be used which drives and operates the pump 22, observes its performance and condition, and/or provides error and status information. The powered controller 1000 may be designed to support at least about 12 hours of continuous operation and can contain a basic interface to indicate and adjust the level of support provided to the patient. Moreover, the controller 1000 may provide an optical and audible alarm notification in case the system detects an error during operation. The controller 1000 may be provided non-sterile and be contained in an enclosure designed for cleaning and re-use outside of the sterile field. The controller 1000 enclosure may contain a socket into which the extension cable is plugged.

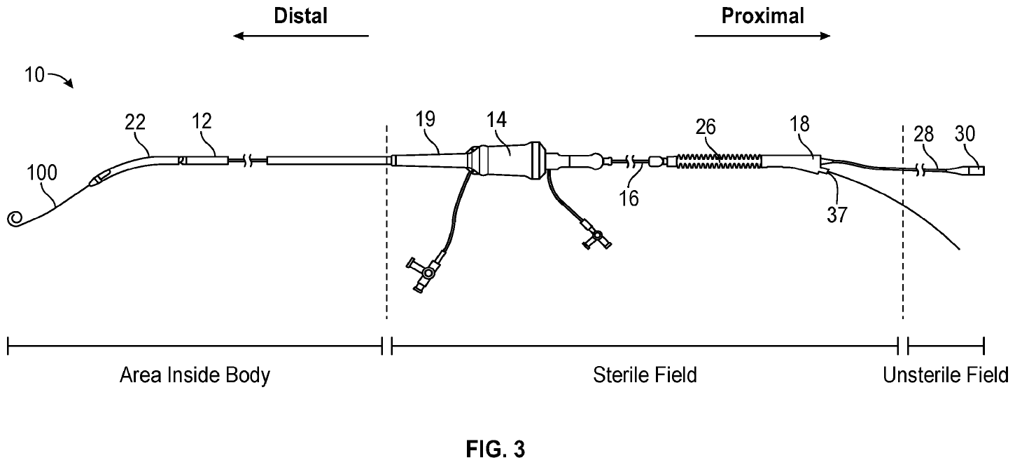

Referring to FIG. 3, there is illustrated an overall MCS system 10 in accordance with some embodiments. For reference, the “distal” and “proximal” directions are indicated by arrows in FIG. 3 and others herein. “Distal” and “proximal” as used herein have their usual and customary meaning, and include, without limitation, a direction more distant from an entry point of the patient's body as measured along the delivery path, and away a direction less distant from an entry point of the patient's body as measured along the delivery path, respectively.

The MCS system 10 may include an introducer sheath 19 having a proximal introducer hub 14 with a central lumen for axially movably receiving an MCS catheter 16 (the MCS catheter 16 may also be referred to as a catheter, catheter shaft, and/or a shaft herein). The catheter 16 may extend between a proximal hub 18 and the pump 22 of the system 10, with a guidewire 100 extending therethrough. An atraumatic cannula tip with, in some embodiments, a radiopaque material allows the implantation/explantation to be visible under fluoroscopy.

The pump 22 comprises a tubular housing. The tubular housing of the pump 22 is used broadly herein and may include any component of the pump 22 or component in the pump region of the system, such as an inlet tube, a distal endpiece, a motor housing 12, other connecting tubular structures, and/or a proximal back end of the motor housing. The pump 22, for example the tubular housing, is carried by a distal region of the catheter 16. The system 10 is provided with at least one central lumen for axially movably receiving the guidewire 100. The proximal hub 18 is additionally provided with an infection shield 26. A proximal cable 28 extends between the proximal hub 18 and a connector 30 for releasable connection to a control system typically outside of the sterile field to drive the pump 22.

In some embodiments, the guidewire or a portion of the guidewire can have a diameter of 0.018″ or about 0.018″. In some embodiments, a diameter of the guidewire may differ at different portions of the guidewire, for example, at a proximal end and/or a distal end. Unless stated otherwise, all measurements (e.g., angles, lengths) described for the guidewire herein are taken along a central axis of the guidewire.

The guidewire 100 can include a distal end or tip that is shaped, dimensioned, and/or otherwise configured to traverse distally through a catheter, for example, a diagnostic catheter having a gauge between 4 Fr and 6 Fr. The catheter may include one or more side holes or openings, for example, for the flushing of fluids (e.g., fluoroscopic fluids) into the aorta/left ventricle of a patient. The distal end or tip of the guidewire 100 may be shaped, dimensioned, and/or otherwise configured to traverse distally through the catheter and out of a distal end of the catheter without exiting the side holes of the catheter.

The guidewire 100 can include a proximal end 104 that is rounded or otherwise shaped, dimensioned, and/or configured to be received within a distal opening of a guidewire lumen of an MCS device or VAD. In some embodiments, a proximal region 108 extending between the proximal end 104 and a transition 107 may be sufficiently soft yet have sufficient column strength and/or axial stiffness to facilitate movement of the guidewire 100 through a non-linear path of the guidewire lumen extending through the MCS or VAD device.

In some embodiments, the guidewire 100 may be designed for movement along sharp or jagged edges, for example of an MCS device or VAD. In some embodiments, the guidewire can be formed of stainless steel, Nitinol, a titanium alloy, a combination thereof, or the like. In comparison to guidewires coated with PTFE or other lubricious materials, some embodiments include guidewires that are uncoated to avoid the scratching or chipping off of the coating when moving along edges of an MCS device or VAD.

In some embodiments, one or more portions of the guidewire 100 can include a coil positioned around an inner core or other features to prevent kinking. In some embodiments, the coil can be formed of Nitinol.

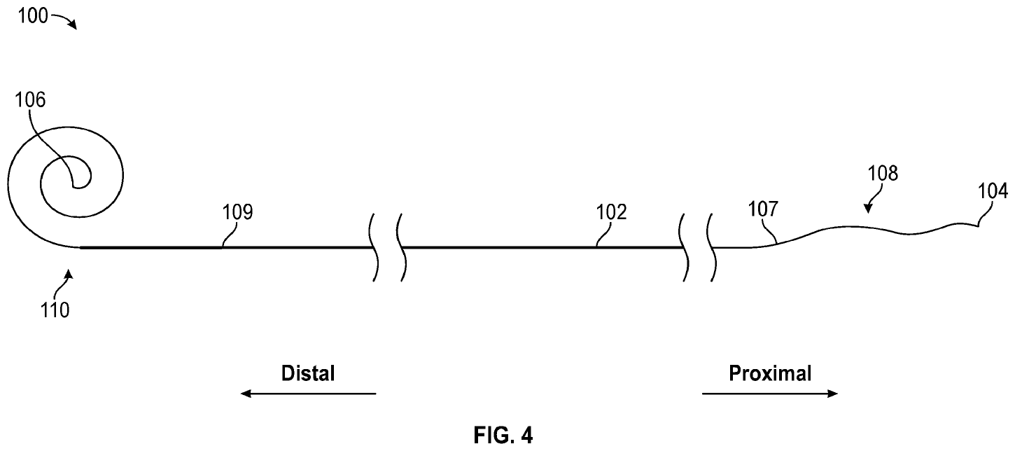

FIG. 4 shows a guidewire 100 having an elongate flexible body 102 extending between a proximal end 104 and a distal end 106. In some embodiments, the guidewire 100 can include a plurality of segments having different diameters. In some embodiments, the guidewire 100 can include stepped or tapered transitions between adjacent segments having different diameters. In some embodiments, the guidewire 100 can include four segments having different diameters. In some embodiments, the guidewire 100 can include tapered transitions between each of the four segments having different diameters. In an alternative embodiment, a transition between a segment having the largest diameter and a segment having the second largest diameter can be a stepped transition.

The guidewire 100 can include a proximal region 108. In some embodiments, the proximal region 108 or a core of the proximal region 108 can have a diameter of 0.18 mm or about 0.18 mm. The proximal region 108 can be configured for threading into and through an MCS device or a VAD. In some embodiments, the proximal region 108 may be sufficiently soft yet have sufficient column strength and/or axial stiffness to facilitate movement of the guidewire 100 relative to a guidewire lumen, for example, of an MCS device or VAD.

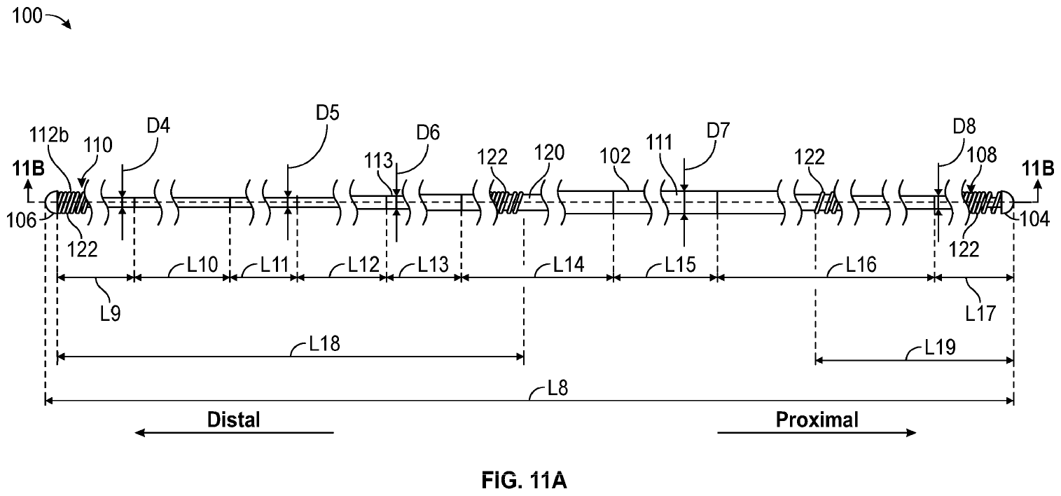

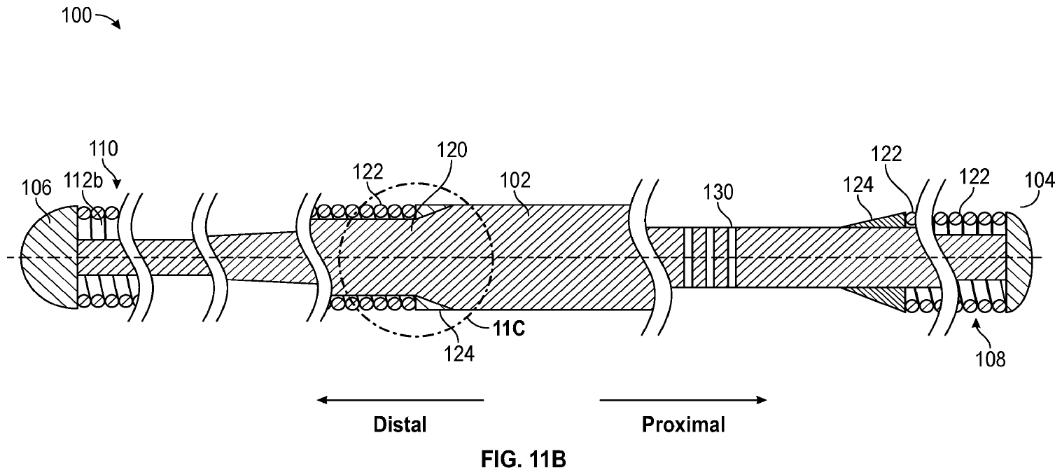

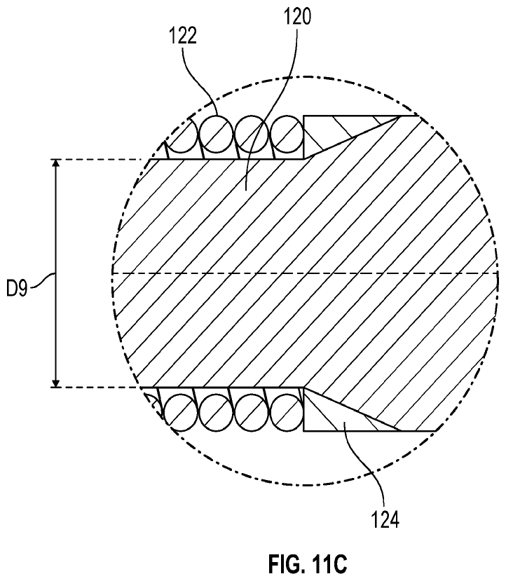

As shown in FIG. 11A, in some embodiments, the body 102 of guidewire 100 includes a body region 111. The body region 111 can be positioned distal to the proximal region 108. In some embodiments, the body region 111 or a core of the body region 111 can have a diameter of 0.47 mm or about 0.47 mm. In some embodiments, the body region 111 may be the least flexible region of the guidewire 100. In some embodiments, the body region 111 forms a majority of the length of the guidewire 100. In some embodiments, the body region 111 is configured to transmit forces (e.g., axial forces) applied when handling the guidewire 100. In some embodiments, the body region 111 can be configured to facilitate an MCS device or VAD passing over the body region 111 during delivery to the heart.

As shown in FIG. 11A, in some embodiments, the body 102 includes a pump region 113. In some embodiments, the pump region 113 can be positioned distal to the body region 111. In some embodiments, the pump region 113 or a core of the pump region 113 can have a diameter of 0.28 mm or about 0.28 mm. In some embodiments, the pump region 113 is configured to be positioned within an MCS device or VAD when the MCS device or VAD is in the heart. In some embodiments, the pump region 113 is less stiff than the body region 111, for example, to more readily traverse bends in the vasculature.

The guidewire 100 can also include a distal region 110. The distal region 110 can be positioned distal to the pump region 113. In some embodiments, the distal region 110 or a core of the distal region 110 can have a diameter of between 0.13 mm or about 0.13 mm and 0.18 mm or about 0.18 mm, or a diameter of 0.14 mm or about 0.14 mm. In some embodiments, the distal region 110 can be the most flexible region of the guidewire 100. In some embodiments, the distal region 110 can be sufficiently flexible to be atraumatic. In some embodiments, the guidewire 100 can be sufficiently flexible to straighten when pulled out of an MCS device or VAD or a catheter shaft, but form a pre-curved and/or pre-bent shape when not constrained (e.g., the guidewire 100 can be resilient).

In some embodiments, an elastic section modulus of the guidewire 100 can be defined by the equation:

$$s = \frac{π {d}^{3}}{32}$$

“S” is the elastic section modulus and “d” is the diameter. For example, in some embodiments, the section modulus S of at least a portion of the proximal region 108 having a diameter of 0.18 mm or about 0.18 mm can be 0.00057 mm³ or about 0.00057 mm³. In some embodiments, the section modulus S of the proximal region 108 can be between 0.0005 mm³ or about 0.0005 mm³ and 0.0006 mm³ or about 0.0006 mm³. In some embodiments, a section modulus S of at least a portion of the body region 111 having a diameter of 0.47 mm or about 0.47 mm can be 0.0102 mm³ or about 0.0102 mm³. In some embodiments, the section modulus of the body region 111 can be between 0.0097 mm³ or about 0.0097 mm³ and 0.0107 mm³ or about 0.0107 mm³. In some embodiments, the section modulus S of at least a portion of the pump region 113 having a diameter of 0.28 mm or about 0.28 mm can be 0.00216 mm³ or about 0.00216 mm³. In some embodiments, the section modulus of the pump region 113 can be between 0.0020 mm³ or about 0.0020 mm³ and 0.0023 mm³ or about 0.0023 mm³. In some embodiments, a section modulus S of at least a portion of the distal region 110 having a diameter of 0.14 mm or about 0.14 mm can be 0.000269 mm³ or about 0.000269 mm³. In some embodiments, a section modulus S of at least a portion of the distal region 110 having a diameter of between 0.13 mm or about 0.13 mm and 0.18 mm or about 0.18 mm can be between 0.000216 mm³ or about 0.000216 mm³ and 0.000573 mm³ or about 0.000573 mm³. In some embodiments, the section modulus S of the distal region 110 can be between 0.00026 mm³ or about 0.00026 mm³ and 0.00028 mm³ or about 0.00028 mm³.

In some embodiments, the proximal region 108 may be more flexible than other regions of the guidewire 100, for example, due to a smaller diameter and/or different chamfers and tapers. In some embodiments, the proximal region 108 may be sufficiently flexible to pass through a guidewire port of an MCS device or VAD having a radius of curvature within a range of from about 5 mm to about 25 mm, and in some embodiments, within a range of from about 10 mm to about 18 mm. In some embodiments, the proximal region 108 may be sufficiently flexible to pass through a guidewire port of an MCS device or VAD having a radius of curvature as small as about 5 mm or as small as about 10 mm. The length of the proximal region 108 between proximal end 104 and a transition 107 may be within a range of from 50 mm or about 50 mm to 500 mm or about 500 mm, within a range of from 60 mm or about 60 mm to 300 mm or about 300 mm, and in some embodiments within a range of from 285 mm or about 285 mm to 295 mm or about 295 mm. The transition region 108 may be a transition between the proximal region 108 and the body region 111.

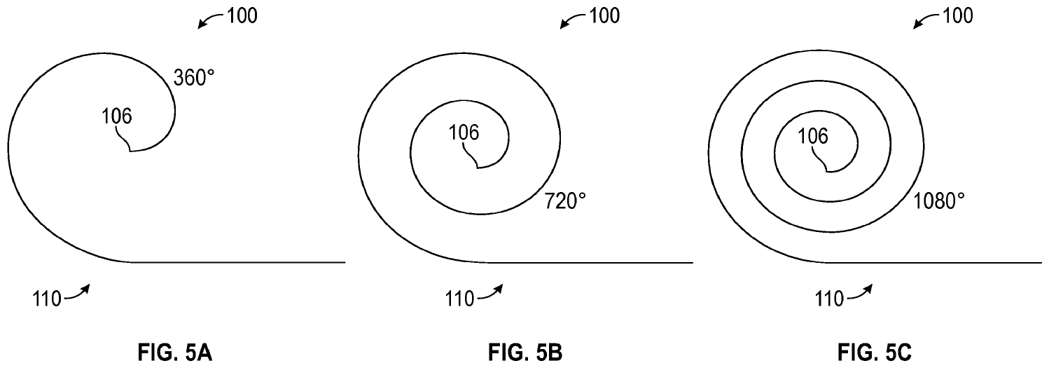

The distal region 110 extending between distal end 106 and a transition 109 may be pre-shaped as a pigtail to provide an atraumatic distal surface. The transition 109 may be a transition between the distal region 110 and the pump region 113. FIGS. 5A-5C depict different embodiments of pigtail configurations of the distal region 110. The pigtail configuration includes a spiral coil geometry in one plane or substantially one plane having an angle of the coil extending between 360° or about 360° and 1080° or about 1080°. For example, the angle of the coil may be 360° or about 360° (as shown in FIG. 5A), 720° or about 720° (as shown in FIG. 5B), or 1080° or about 1080° (as shown in FIG. 5C). In some embodiments, the spiral coil geometry can be defined by a section of the distal region 110 in the shape of a spiral. In some embodiments, a spiral coil geometry can include a curve that winds around a central point at a continuously increasing distance from the central point or at a continuously decreasing distance towards the central point. In some embodiments, a spiral coil geometry can include a continuous and widening curve extending from and about a central point or a continuous and tightening curve extending towards and about a central point. In some embodiments, the spiral coil geometry can include a plurality of loops having differing diameters. All of the coils of the spiral coil may be in a plane or substantially within a plane.

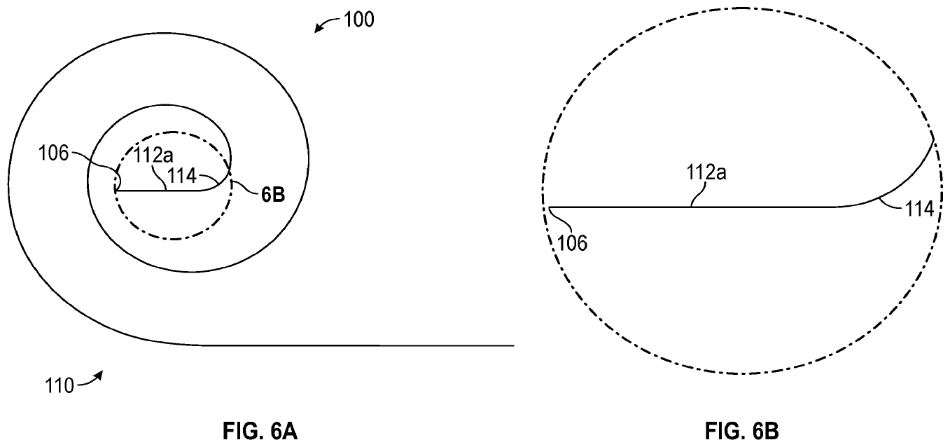

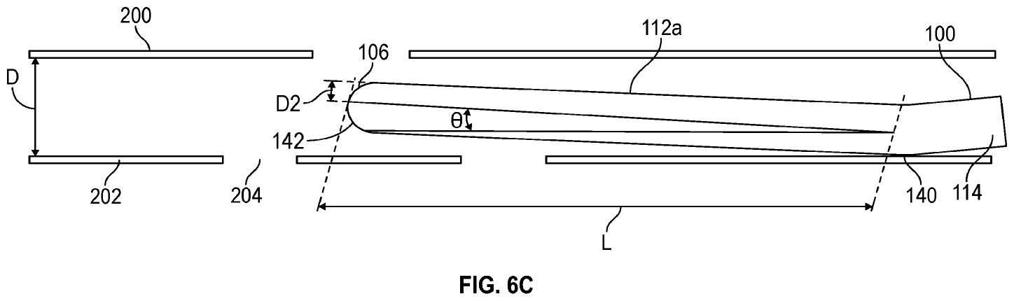

FIGS. 6A-6B depict an embodiment of a distal region 110 including a distal advance segment 112a. The advance segment 112a can be in the form of an elongated straight tip extending from an inflection point between a proximal end of the advance segment 112a and a distal end 114 of the pigtail configuration. The elongated advance segment 112a can be shaped, dimensioned, or otherwise configured to traverse distally through a catheter and out of a distal end of the catheter without exiting side holes of the catheter. The elongated advance segment 112a may have a length of between 0.5 mm or about 0.5 mm and 25 mm or about 25 mm, between 1 mm or about 1 mm and 25 mm or about 25 mm, or between 2 mm or about 2 mm and 25 mm or about 25 mm. The length of the advance segment 112a may prevent or inhibit the advance segment 112a from exiting side holes of a catheter. In some embodiments, an angle of the inflection point between the distal end 114 of the pigtail configuration and the elongated advance segment 112a is sufficiently small to, in combination with the length of the advance segment 112a, prevent the distal end 106 and/or a distal end 142 of the advance segment 112a (as shown in FIG. 6C) from exiting the side holes of the catheter. For example, in some embodiments, the angle of the inflection point between the distal end 114 of the pigtail configuration and the elongated advance segment 112a is within a range of from 5° or about 5° to 85° or about 85°.

FIG. 6C depicts an example of the distal advance segment 112a within a catheter 200. In some embodiments, the catheter 200 can be a 4 F or a 5 F catheter. The catheter 200 can have an inner diameter D between 0.9 mm or about 0.9 mm and 1.2 mm or about 1.2 mm. In some embodiments, the catheter 200 can have an inner diameter of 1.07mm or about 1.07 mm, a diameter of 1.19 mm or about 1.19 mm, or any other suitable inner diameter, D. The catheter 200 can include sidewalls 202 having one or more side holes 204. The side holes 204 can have a diameter of between 0.5 mm or about 0.5 mm and 0.8 mm or about 0.8 mm. In some embodiments, the catheter 200 can have between 1 and 16 side holes 204, between 4 and 12 side holes 204, or any other suitable range. In some embodiments, the catheter 200 can have 8 side holes 204.

As described herein, the length L of the advance segment 112a can prevent or inhibit the elongated advance segment 112a from exiting the side holes 204. For example, the length L of the advance segment 112a can be greater than the maximum diameter of the side holes 204 to an extent that prevents the distal end 106 of the guidewire 100 and/or the distal end 142 of the advance segment 112a from exiting the side holes 204. As shown in FIG. 6C, an angle between a distal end 114 of the pigtail configuration and the advance segment 112a can be sufficiently small such that when the inflection point at the proximal end 140 of advance segment 112a contacts a sidewall 202, the distal end 106 of the guidewire 100 and/or the distal end 142 of the advance segment 112a can be located at or near a radial center of the catheter 200, and/or at an angle to a plane of the side hole that prevents the distal end 106 of the guidewire 100 and/or the distal end 142 of the advance segment 112a from entering the side hole 204.

The length L of the advance segment 112a and the angle at the inflection point where the advance segment 112a extends from the distal end 114 of the pigtail configuration can thus be sized such that no more than a portion D2 of a diameter of the distal end 106 can extend into one of the side holes 204. The portion D2 can be less than half or about half of the diameter of the distal end 106 such that contact of the distal end 106 with an edge of one of the side holes 204 while the portion D2 is within the side hole 204 will result in the distal end 106 deflecting into the catheter 200 and not out of the side hole 204 or not being stuck in the side hole 204. In some embodiments, the portion D2 can be less than one third or about one third or less than one quarter or about one quarter of the diameter of the distal end 106. In some embodiments, the portion D2 of the diameter can be less than 0.1524 mm or about 0.1524 mm or less than 0.1143 mm or about 0.1143 mm. In some embodiments, the portion D2 of the diameter can be within a range of from 0.05 mm or about 0.05 mm to 0.23 mm or about 0.23 mm, 0.1 mm or about 0.1 mm to 0.18 mm or about 0.18 mm, 0.1 mm or about 0.1 mm to 0.16 mm or about 0.16 mm, 0.15 mm or about 0.15 mm to 0.23 mm or about 0.23 mm, or any other suitable range. In some embodiments, a minimum length L of the advance segment 112a may be within a range of between 0.5 mm or about 0.5 mm to 3.5 mm or about 3.5 mm or between 1.58 mm or about 1.58 mm and 3.318 mm or about 3.318 mm. In some embodiments, the guidewire 100 can be configured such that an angle θ between a central axis of the advance segment 112a and a longitudinal axis of the catheter can have a maximum within a range of between 17° or about 17° and 25° or about 25°.

The distal end 106 of the guidewire 100 and/or the distal end 142 of the advance segment 112a can be rounded in shape. For example, the distal end 106 and/or distal end 142 can be hemispherical, hemispheroidal, parabolic, or otherwise convex in shape. A rounded shape of the distal end 106 and/or distal end 142 can cause the distal end 106 and/or distal end 142 to roll or deflect off of an edge of one of the side holes 204 if the distal end 106 and/or distal end 142 comes into contact with such an edge of one of the side holes 204.

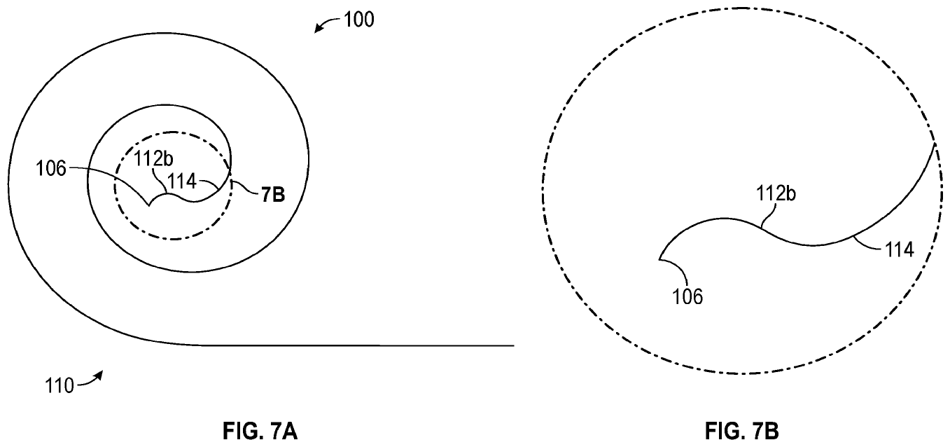

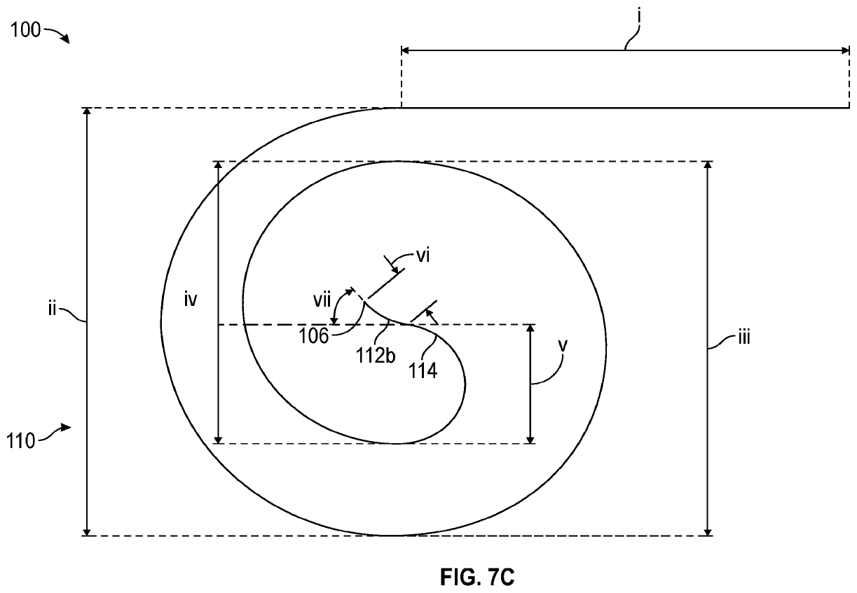

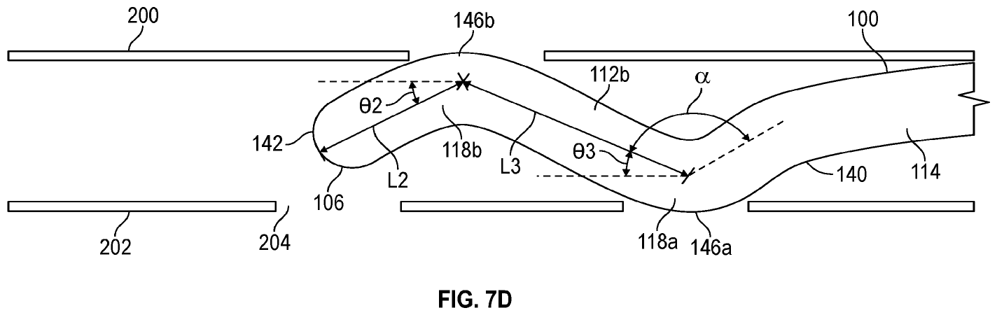

FIGS. 7A-7B depict an embodiment of a distal region 110 including a curved advance segment 112b. The advance segment 112b can be in the form of a curved or s-shaped segment extending from an inflection point at the distal end 114 of the pigtail configuration. The advance segment 112b can be shaped, dimensioned, or otherwise configured to traverse distally through the catheter and out of a distal end of the catheter without exiting the side holes of the catheter. In the illustrated implementation, the pigtail configuration is concave in a first direction which ends at the transition with the curved advance segment 112b which is concave in a second, opposite direction. This curvature may reduce the risk of scratching and reduce friction when the guidewire is traversed through a catheter and/or a guidewire lumen.

FIG. 7C depicts an example of an embodiment of a distal region 110 having a curved advance segment 112b indicating examples of dimensions for portions of the distal region 110. In some embodiments, a length i of a portion of the distal region 110 can be 79 mm or about 79 mm. A diameter ii can be 30 mm or about 30 mm. A diameter iii can be 26 mm or about 26 mm. A diameter iv can be 20 mm or about 20 mm. A diameter v can be 7 mm or about 7 mm. The pigtail region, if straightened, can have a length of 115 mm or about 115 mm, 160 mm or about 160 mm, 210 mm or about 210 mm, between 100 mm or about 100 mm and 175 mm or about 175 mm, between 100 mm or about 100 mm and 225 mm or about 225 mm, or any other suitable length.

In some embodiments, the curved/bent advance segment 112b can have a length vi of 1.0 mm, about 1.0 mm, 0.5 mm, about 0.5 mm, 1.5 mm, about 1.5 mm, or between 0.5 mm and 1.5 mm. The curved/bent advance segment 112b can incline at an angle vii of 10°, about 10°, 15°, about 15°, 20°, about 20°, 25°, about 25°, 30°, about 30°, 60°,about 60°, between 10° or about 10° and 60° or about 60°, between 15° or about 15° and 60° or about 60°, or any other suitable angle or range of angles relative to a tangent line of a distal end 114 of the pigtail configuration. The length vi (for example, 1.0 mm length) and angle vii (for example, 10° or about 10° to 60° or about 60° angle) may prevent the distal end 106 of the guidewire 100 and/or a distal end 142 of the advance segment 112b from contacting a sidewall of the catheter when an inflection point or region around the inflection point contacts the sidewall of the catheter while the guidewire 100 traverses through the catheter 200, for example as described with respect to FIG. 7D. In some embodiments, the length vi (for example, 1.0 mm or about 1.0 mm length) and angle vii (for example, 10° or about 10° to 60° or about 60° angle) may cause the distal end 106 and/or distal end 142 to be spaced apart from the inside surface of the central lumen such as in the vicinity of the radial center of the catheter 200 when an inflection point or region around the inflection point contacts the sidewall 202 of the catheter 200.

FIG. 7D depicts an example of the advance segment 112b within the catheter 200. As shown in FIG. 7D, the advance segment 112b can be s-shaped having a first curved region 118a with an inflection point 146a and a second curved region 118b with an inflection point 146b. In some embodiments, the curved region 118a and/or 118b can have an arc length greater than the diameter of the side holes 204 to prevent or restrict the advance segment 112b from exiting the side holes 204. In some embodiments, the arc length may be greater than 0.5 mm or about 0.5 mm or greater than 0.8 mm or about 0.8 mm. In some embodiments, the arc length can be within a range of from 0.5 mm or about 0.5 mm to 1.5 mm or about 1.5 mm, or within a range of from 0.8 mm or about 0.8 mm to 1.5 mm or about 1.5 mm. In some embodiments, the inflection point 146a and/or the inflection point 146b can be configured to contact and/or slide along the sidewalls 202 as the guidewire 100 traverses through the catheter 200.

A portion of the advance segment 112b can extend from the inflection point 146b at an angle and over a length that prevent the distal end 106 of the guidewire 100 and/or the distal end 142 of the advance segment 112b from contacting the sidewalls 202 while the guidewire traverses within the catheter 200. For example, the curvature and length of the section of the advance segment 112b extending from the inflection point 146b can cause the distal end 106 of the guidewire 100 and/or the distal end 142 of the advance segment 112b to be located at or near a radial center of the catheter 200 when the inflection point 146b or a region around the inflection point 146b contacts the sidewall 202. By locating the distal end 106 and/or distal end 142 at or adjacent the radial center, the curved shape of the advance segment 112b can prevent or restrict the advance segment 112b from exiting the side holes 204. In some embodiments, the catheter 100 can be configured such that a portion of the advance segment 112b can extend from the inflection point 146b at an angle θ2 between a central axis of the portion of the advance segment 112b and a longitudinal axis of the catheter 200. The angle θ2 can have a maximum within a range of from 5° or about 5° to 85° or about 85°, or from 30° or about 30° to 60° or about 60°. In some embodiments, a portion of the advance segment 112b can extend from the inflection point 146b over a length L2 measured along a central axis of the portion of the advance segment 112b. The length L2 can be within a range of from 0.3 mm or about 0.3 mm to 4 mm or about 4 mm, from 0.3 mm or about 0.3 mm to 1.5 mm or about 1.5 mm, or from 1 mm or about 1 mm to 4 mm or about 4 mm. The curvature at the inflection points 146a and 146b and the distance in between can cause one or both of the inflection points 146a and 146b to contact opposing sidewalls 202 of the catheter 200. In some embodiments, a radius of curvature at at least one of or both of the inflection points 146a and 146b can be greater than a radius of the side holes 204. For example, in some embodiments, the radius of curvature at the inflection points 146a and 146b can be within a range of 0.5 mm or about 0.5 mm to 0.8 mm or about 0.8 mm. In some embodiments, the curved region 118a can be defined by an angle a extending between a central axis of the curved region 118a proximal to the inflection point 146a and a central axis of the curved region 118a distal the inflection point 146a. In some embodiments, the angle α can be within a range of between 10° or about 10° and 70° or about 170°, or between 60° or about 60° and 120° or about 120°. In some embodiments, a length L3 taken along a central axis of the segment between the inflection point 146a and the inflection point 146b has a minimum defined by the equation:

$$L 3 min = \frac{0.43}{sin ( \frac{180 - α}{2} )}$$

L3 min can be the length that causes the inflection points 146a and 146b to contact the sidewalls 202 of the catheter 200 when the catheter 200 has a diameter of 0.9 mm or about 0.9 mm.

In some embodiments, the length L3 can have a maximum defined by the equation:

$$L 3 max = \frac{0.73}{sin ( \frac{180 - α}{2} )}$$

L3 max can be the length that causes the inflection points 146a and 146b to contact the sidewalls 202 of the catheter 200 when the catheter 200 has a diameter of 1.2 mm or about 1.2 mm.

In an embodiment in which the angle α is 140° or about 140°, the length L3 can be in a range of between 1.257 mm or about 1.257 mm and 2.13 mm or about 2.13 mm. In some embodiments, an angle θ2 between a central axis of the segment between the inflection point 146a and the inflection point 146b and a longitudinal axis of the catheter can be within a range of from 5° or about 5° to 85° or about 85°, or from 30° or about 30° to 60° or about 60°. In some embodiments, if the length L2 is less than the length L3, and the angle θ2 has the same or about the same magnitude as the angle θ3, the distal end 106 and/or distal end 142 will not extend to the opposing sidewall 202, preventing or restricting the distal end 106 and/or distal end 142 from exiting a side hole 204.

The distal end 106 of the guidewire 100 and/or the distal end 142 of the advance segment 112b can be rounded in shape. For example, the distal end 106 and/or distal end 142 can be hemispherical, hemispheroidal, parabolic, or otherwise convex in shape. A rounded shape of the distal end 106 and/or distal end 142 can cause the distal end 106 and/or distal end 142 to roll or deflect off of an edge of one of the side holes 204 if the distal end 106 and/or distal end 142 comes into contact with such an edge of one of the side holes 204.

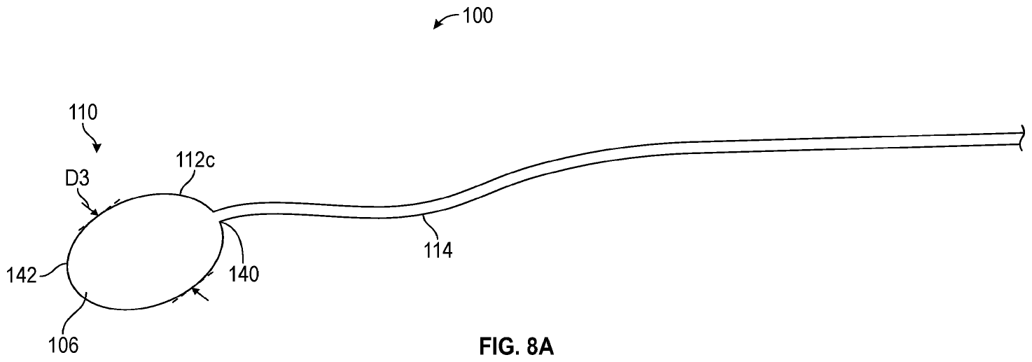

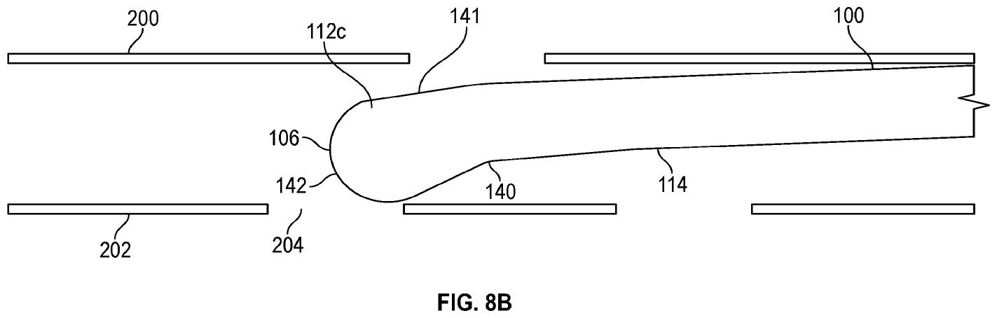

FIG. 8A depicts an embodiment of a distal region 110 in which the distal end 106 includes a distal advance segment 112c in the form of an enlarged distal tip. The advance segment 112c can be shaped, dimensioned, or otherwise configured to traverse distally through the catheter and out of a distal end of the catheter without exiting the side holes of the catheter. The advance segment 112c can have a diameter D3 that is larger than the portion of the pigtail region immediately proximal to the advance segment 112c. For example, the advance segment 112c or a maximum cross-section of the advance segment 112c can have a diameter D3 of between 0.8 mm or about 0.8 mm and 1 mm or about 1 mm. In some embodiments, the advance segment 112c can be spherical or spheroidal in shape, for example, as shown in FIG. 8A.

FIG. 8B depicts an example of an advance segment 112c with an enlarged distal tip within the catheter 200. In some embodiments a diameter at a largest cross-section of the advance segment can be greater than or equal to the diameter of the side holes 204 to prevent the advance segment 112c from exiting the side holes 204. As described above, the diameter at the largest cross-section of the advance segment can be between 0.8 mm or about 0.8 mm and 1 mm or about 1 mm, and the diameter of the side holes 204 can be between 0.5 mm or about 0.5 mm and 0.8 mm or about 0.8 mm.

As shown in FIG. 8B, in some embodiments, the distal end 106 of the guidewire 100 and/or the distal end 142 of the advance segment 112c can have a rounded shape that tapers proximally over a tapered portion 141 from the distal end 106 and/or distal end 142 to a proximal end 140 of the advance segment 112c. The distal end 106 and/or distal end 142 can be hemispherical, hemispheroidal, parabolic, or otherwise convex in shape. A rounded shape of the distal end 106 and/or distal end 142 can cause the distal end 106 to roll or deflect off of an edge of one of the side holes 204 if the distal end 106 and/or distal end 142 comes into contact with such an edge of one of the side holes 204. In some embodiments, the tapered shape of the tapered portion 141 can prevent catching of the guidewire on edges of a catheter/inlet tube of an MCS device and/or VAD when removed therefrom.

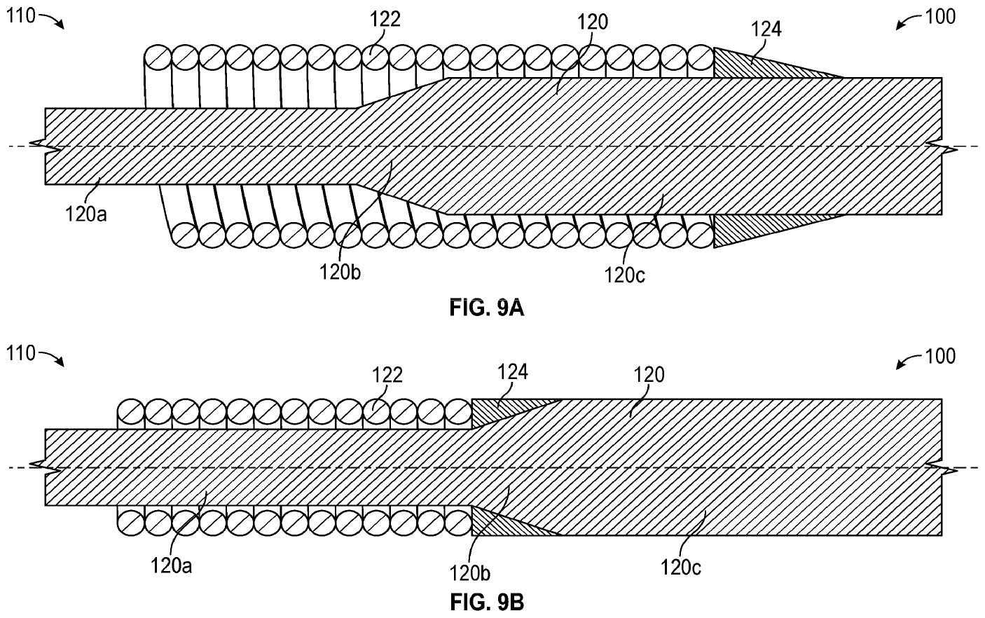

FIGS. 9A-9B depict embodiments of a portion of the distal region 110. In some embodiments, at least part of the distal region 110 and/or the elongate flexible body 102 can include a core 120 and a coil 122 of thin wire surrounding the core 120. The coil 122 can help prevent the guidewire 100 from kinking. As shown in FIGS. 9A-9B, the core 120 may include segments 120a and 120c having different diameters. In some embodiments, the diameter of the core 120 can be between 0.10 mm or about 0.10 mm and 0.5 mm or about 0.5 mm. In some embodiments, the diameter of the core 120 can be between 0.13 mm or about 0.13 mm and 0.18 mm or about 0.18 mm. In some embodiments, the diameter of the core 120 can be between 0.43 mm or about 0.43 mm and 0.5 mm or about 0.5 mm. In some embodiments, the maximum diameter of the guidewire 100 can be 0.5 mm or about 0.5 mm, for example, to allow for the use of catheters not exceeding 10 Fr.

The segments 120a and 120c can be connected by a tapered or chamfered transition segment 120b. The different diameters and tapered or chamfered segment can provide for different levels of flexibility between the different segments. For example, segment 120a, which is positioned distally to segment 120c, can have increased flexibility in comparison to the segment 120c due to its smaller diameter and the tapered or chamfered connection. In this way, the distal region 110 and/or distal end 106 can be more flexible than more proximal portions of the guidewire 100.

In some embodiments, the coil 122 can be soldered to the core 120 using solder 124. FIG. 9A depicts the coil 122 extending over the segment 120a, the tapered or chamfered transition segment 120b and the segment 120c. The coil 122 is soldered to the segment 120c in FIG. 9A. As shown in FIG. 9A, the solder 124 can be in the form of a chamfer to provide for a smooth transition. By extending across the transition segment 120b, the arrangement of the coil 122 shown in FIG. 9A may prevent kinking at the transition segment 120b. Such an arrangement may allow for a segment 120a having a smaller diameter and increased softness and/or flexibility. In FIG. 9B, the coil extends over the segment 120a and is soldered to the tapered transition segment 120b. As shown in FIG. 9B, the coil 122 has the same or about the same outer diameter as the segment 120c. The solder 124 can be applied so that the outer diameter of the segment 120c, the coil 122, and the solder 124 have a continuous or substantially continuous and smooth outer diameter. This arrangement avoids edges that may catch during movement of the guidewire 100. In other embodiments, the coil 122 can be glued, welded, or otherwise adhered to the core 120.

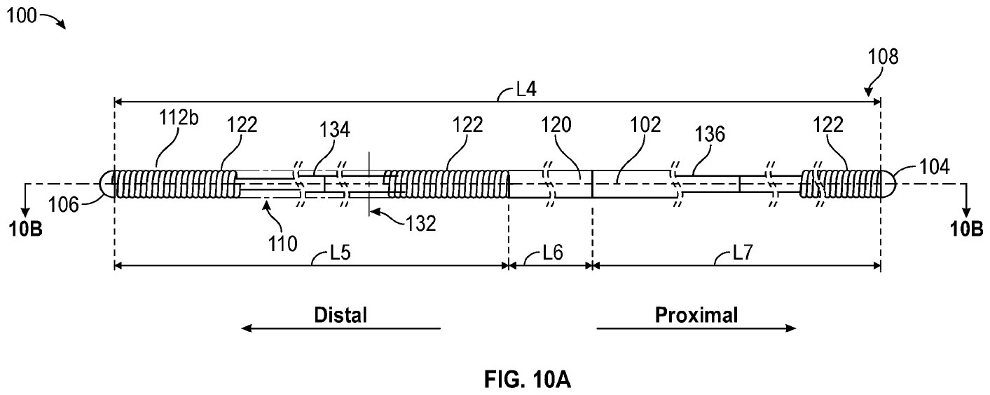

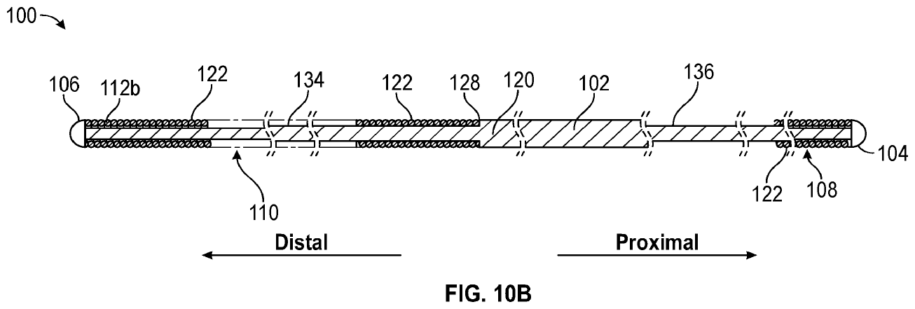

While FIGS. 9A-9B show the distal region 110, the use of coils and/or tapered or chamfered segments to affect the flexibility of different regions of the guidewire 100 can be used in at least part of the other regions as well, such as the proximal region 108. the body region 111, and/or the pump region 113, for example, as shown in FIGS. 10A-10B.

FIGS. 10A-10B depict an embodiment of a guidewire 100. As shown in FIGS. 10A-10B, the proximal end 104 and the distal end 106 can include rounded edges. Rounded edges of the distal end 106 can provide for atraumatic movement of the distal end 106 within the anatomy. Rounded edges of the proximal end 104 can facilitate insertion of the proximal end 104 into an MCS device and/or VAD and their respective catheter 16. The rounded (for example, hemispherical) shape of the distal end 106 and/or proximal end 104 can be formed by forming, by machining, by welding and/or by melting the core 120 and optionally the coils 122. Alternatively, the core 120 and optionally coils 122 can be soldered to form the rounded distal end 106. Alternatively, a glue bead can be used to form the rounded end and be adhered to the core 120 and optionally the coils 122.

As shown in FIGS. 10A-10B, the guidewire 100 can include coils 122 surrounding the core 120 at the proximal region 108 and/or distal region 110. The core 120 at the proximal and/or distal regions can have a smaller diameter than the core 120 at more central regions of the guidewire 100. For example, in FIG. 10B the diameter of the core 120 can be about 0.18 mm at the proximal end 104 and at the distal end 106, but about 0.457 mm at a central region of the guidewire. The smaller diameter at the proximal end 104 and/or distal end 106 can provide for increased flexibility at the proximal end 104 and/or the distal end 106. The coils 122 can prevent kinking in these regions having smaller diameters. As shown in FIGS. 10A-10B, in some embodiments, the coils 122 can be glued to the core 120.

In some embodiments, the distal region 110 can be made radiopaque along its entire length, such as with a discrete plurality of markers or with a continuous marker. For example, a discrete radiopaque marker can include a gold solder at the distal tip 106, a gold solder 124 connecting a coil 122 to the core 120, and/or a plurality of radiopaque spots spaced (e.g., evenly spaced) along the length of the distal region 110. Alternatively, or in addition, at least a portion of the guidewire 100 (e.g., the distal region 110 or at least a portion thereof) can have a polymer coating doped with barium sulfate or another radiopaque material. In some embodiments, a coil 122 of the distal region 110 can be made from a radiopaque material or metal such as platinum, gold, iridium, or a combination thereof. In use, a radiopaque distal region 110 may be seen on fluoroscopy without the need to inject a contrast agent. This can advantageously facilitate case of use and safety by allowing the physician/user to see and confirm that the distal region 110 is properly positioned in the cardiovascular system of the patient (e.g., in the left ventricle) or if it has migrated out and needs attention to reposition. Additionally, this can also help prevent tangling of the guidewire 100 with anatomical structures or with other medical components near the guidewire 100. In other embodiments, the distal region 110 can be made radiopaque along a portion of its length. For example, the distal region 110 can be radiopaque along an advance segment or at least a portion of an advance segment.

In some embodiments, the core 120 can include one or more stepped transitions 128 between adjacent segments having different diameters. As shown in FIG. 10B, in some embodiments, a coil 122 can be wrapped around segment(s) of the core 120 having a smaller diameter and abut a stepped transition to reduce or prevent the guidewire 100 from having sharp edges that could catch on or scratch other components. The core 120 can also include one or more tapered or chamfered transition segments, which can be conical or frustoconical, between adjacent segments having different diameters. For example, the guidewire 100 can include a conical or frustoconical transition segment 134 in the distal portion 110 and/or a conical or frustoconical transition segment 136 in or adjacent the proximal portion 108.

As shown in FIGS. 10A-10B, the overall length L4 of the guidewire 100 (not including the rounded proximal end 104 and distal end 106) can be 3000 mm±30 mm or about 3000 mm±30 mm. A length L5 of the distal region 110 of the guidewire 100 can be 135 mm±35 mm or about 135 mm±35 mm and at least a portion may be configured as a pigtail as described herein beginning at a point 132 along the length L5. A length L6 of the central region of the guidewire 100 can be 2600 mm±300 mm or about 2600 mm±300 mm. A length L7 of the proximal region 108 of the guidewire 100 can be 328 mm±50 mm or about 328 mm±50 mm.

FIGS. 11A-11C depict another embodiment of a guidewire 100. As described with respect to FIGS. 10A-10B, the proximal region 108 and/or the distal region 110 of the core 120 can have a smaller diameter than central regions of the guidewire 100 for increased flexibility. The proximal region 108 and/or the distal region 110 can include a coil 122 surrounding the core 120 to prevent kinking.

As shown in FIG. 11A, the guidewire 100 can include one or more tapered segments to transition between segments having different diameters. As shown in FIGS. 11B-11C, in some embodiments, solder 124 can be used to attach the coils 122 to the core 120. In some embodiments, the solder 124 can provide for a smooth transition between the coil 122 and adjacent segments to reduce or prevent sharp edges.

As shown in FIG. 11A, the overall length L8 of the guidewire 100 can be 3000 mm±20 mm or about 3000 mm±20 mm. A length L9 of the distal region 110 of the guidewire 100 can be 38 mm±3 mm or about 38 mm±3 mm and have a diameter D4 of 0.130 mm±0.004 mm or about 0.130 mm±0.004 mm, 0.180 mm±0.004 mm or about 0.180 mm±0.004 mm, or between 0.130 mm±0.004 mm or about 0.130 mm±0.004 mm and 0.180 mm±0.004 mm or about 0.180 mm±0.004 mm. A length L11 proximal to the length L9 can be 30 mm±3 mm or about 30 mm±3 mm and have a diameter D5 of 0.237±0.004 mm or about 0.237±0.004 mm. A length L10 of the guidewire 100 in between the lengths L9 and L11 can be tapered across its length of 20 mm±3 mm or about 20 mm±3 mm and transition from the diameter D4 at its distal end to the diameter D5 at its proximal end. A length L13 of the pump region 113 of the guidewire 100 can be 35 mm±3 mm or about 35 mm±3 mm and have a diameter D6 of 0.280±0.004 mm or about 0.280±0.004 mm. A length L12 of the guidewire 100 in between the lengths L11 and L13 can be tapered across its length of 30 mm±3 mm or about 30 mm±3 mm and transition from the diameter D5 at its distal end to the diameter D6 at its proximal end. A length L15 of the body region 111 of the guidewire 100 can be 2427 mm±20 mm or about 2427 mm±20 mm and have a diameter D7 of 0.470 mm±0.005 mm or about 0.470 mm±0.005 mm. A length L14 of the guidewire 100 in between the lengths L13 and L15 can be tapered across its length of 50 mm±3 mm or about 50 mm±3 mm and transition from the diameter D6 at its distal end to the diameter D7 at its proximal end. A length L17 of the proximal region 108 of the guidewire 100 can be 40 mm±3 mm or about 40 mm±3 mm and have a diameter D8 of 0.130 mm±0.004 mm or about 0.130 mm±0.004 mm. A length L16 of the guidewire 100 in between the lengths L15 and L17 can be tapered across its length of 330 mm±6 mm or about 330 mm±6 mm and transition from the diameter D7 at its distal end to the diameter D8 at its proximal end. In some embodiments, a coil 122 can attach to the core 120 and extend distally from a length L18 of 160 mm±5 mm or about 160 mm±5 mm from the distal end 106. In some embodiments, a coil 122 can attach to the core 120 and extend proximally from a length L19 of 300 mm±5 mm or about 300 mm±5 mm from the proximal end 104. In some embodiments, the length L16 can include a tapered section at its distal end with a length of 80 mm±3 mm or about 80 mm±3 mm that transitions from the diameter D7 to a diameter of 0.241 mm±0.004 mm or about 0.241 mm±0.004 mm, and a tapered section at its proximal end with a length of 250 mm±3 mm or about 250 mm±3 mm that transitions from the diameter of 0.241 mm±0.004 mm or about 0.241 mm±0.004 mm to the diameter D8.

As shown in FIG. 11B, the elongate flexible body 102 can include one or more markers 130. In some embodiments, the marker(s) 130 can be radiopaque markers. In some embodiments, the marker(s) 130 can be visually distinguishable when looking at the guidewire 100 (e.g., by the naked eye without fluoroscopy) and can comprise a metal band, ink, heat shrink, be etched, grinded, soldered, and/or a welded metal layer. The marker(s) 130 can be positioned 15 mm or about 15 mm distal to a coil end point of the adjacent coil 122. The marker(s) 130 may be 1 mm or about 1 mm in width and spaced apart at 2 mm or about 2 mm intervals, although other dimensions are considered within the scope of this disclosure. In some embodiments and as shown in FIG. 11B, the guidewire 100 can include three markers 130, although any number of markers 130 can be included. While the marker(s) 130 are shown positioned at a particular body segment of the guidewire in FIG. 11B, in certain embodiments, one or more marker(s) 130 can be positioned at any location along the guidewire 100. In certain embodiments, the guidewire 100 can include markers 130 positioned on a plurality of different segments of the guidewire 100. For example, the guidewire can include a first marker 130 or first plurality of markers 130 on a first segment of the guidewire 100 and a second marker 130 or second plurality of markers 130 on a second segment of the guidewire 100.

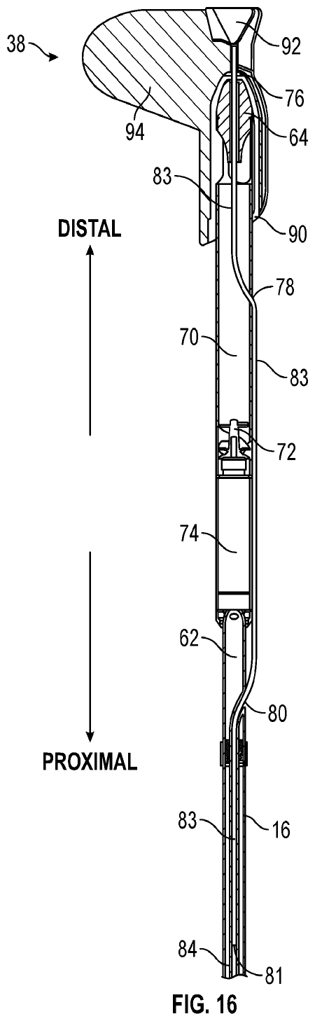

The marker(s) 130 can be positioned to provide an indication for a user that the guidewire 100 has advanced to a sufficient extent within an MCS device or VAD when aligned with an additional reference point while advancing the guidewire 100 into the MCS device or VAD. For example, as described with respect to FIG. 16, a removable guidewire aid 38 can be used with the MCS device or VAD to assist in positioning the guidewire 100 therein. The marker(s) 130 can be positioned so as to align with a distal end of the guidewire aid 38 to indicate that the guidewire 100 has advanced to a sufficient extent so as to be secure within the MCS device or VAD (for example, within the catheter 16) such that the guidewire aid 38 can be removed. In some embodiments, a distance between the markers 130 and the proximal end 104 can be greater than a length of the guidewire aid 38.

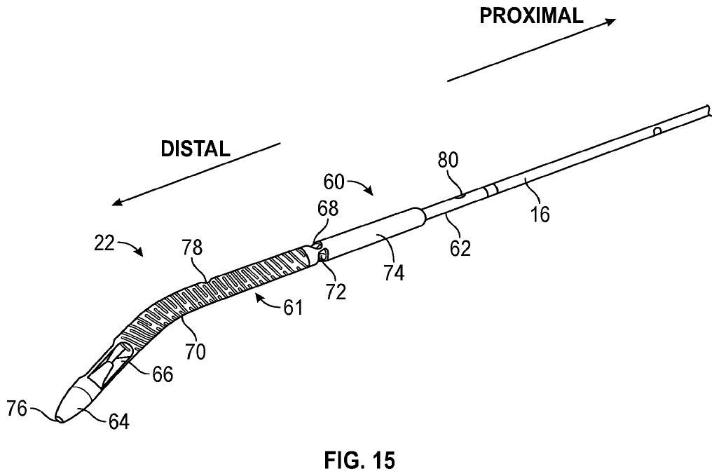

In some embodiments, one or more marker(s) 130 can be located on the guidewire 100 at a distance X from the distal end 106 of the guidewire 100 such that when the distal end 106 is aligned with a guidewire port of the MCS shaft (e.g., the third guidewire port 80 described with respect to FIGS. 15-21), the marker(s) 130 are just exiting (for example, immediately proximal to) the proximal guidewire port 37 of the proximal hub 18 of the MCS system (with reference to FIG. 3). For example, the distance X can be equal to or about equal to the distance between the guidewire port of the MCS shaft (e.g., the third guidewire port 80 described with respect to FIGS. 15-21) to the proximal guidewire port 37 of the proximal hub 18 of the MCS system, plus an amount Y, wherein Y can be in the range of 0 mm or about 0 mm to 10 mm or about 10 mm, preferably 2 mm or about 2 mm. In some embodiments, the marker(s) 130 can have an overall length (along the longitudinal axis of the guidewire 100) in a range of 1 mm or about 1 mm to 10 mm or about 10 mm, preferably 5 mm or about 5 mm. In use, a physician/user may have an MCS device with the guidewire 100 passing therethrough such that the distal tip 106 of the guidewire 100 is further distal than a distal guidewire port of the MCS device (e.g., the first guidewire port 76 described with respect to FIG. 16), such as when positioned in the left ventricle 93 as shown in FIG. 1B. When the physician/user desires to retract the guidewire, the guidewire 100 and the proximal hub 18 of the MCS system, both external to the patient, may be visible to the physician/user while retracting. When the marker(s) 130 appear to come out of the proximal hub 18 (e.g., out of the proximal guidewire port 37) it can serve as an indication that the distal tip 106 of the guidewire 100 is no longer protruding distally from the MCS device (e.g., the distal tip 106 is within the MCS device, its relief bend 62 with reference to FIG. 16, or its catheter 16). In some embodiments, the guidewire 100 can include marker(s) 130, which may be optically distinguishable from one another, placed at distances from the distal tip 106 of the guidewire 100 such that they align with the exit (e.g., proximal guidewire port 37) of the proximal hub 18 of the MCS system (and can therefore be seen by the physician/user) when the distal tip 106 of the guidewire 100 is aligned with various guidewire ports of the MCS system, such as the first guidewire port 76, the second guidewire port 78, and/or the third guidewire port 80 as described with respect to FIG. 16.

FIG. 11C shows a sectional view of the cross-section of the guidewire of FIG. 11B. As discussed previously, coil 122 can be soldered to the core 120 using solder 124 (e.g., soldered circumferentially at a longitudinal end of the coil 122 as shown). Furthermore, the coil 122 can have the same or about the same outer diameter as the solder 124. To accomplish this, the diameter of the core, such as diameter D9 shown in FIG. 11C, can be reduced. For example, the core can have a diameter D9 of 0.270 mm±0.004 mm or about 0.270 mm±0.004 mm to allow for the coil 122 to wrap therearound and have an overall diameter of 0.47 mm±0.005 mm or about 0.47 mm±0.005 mm. Coils 122, when included in the guidewire 100, can have a wire thickness of between 0.078 mm or about 0.078 mm to 0.082 mm or about 0.082 mm. In some embodiments, multiple coils 122 can be used with the guidewire 100 over different sections or segments of the guidewire 100. Furthermore, multiple coils 122 can have different wire thicknesses and outer diameters. The coil 122 may be stainless steel, nitinol, or the like, or may be made of a radiopaque material as described herein. In some embodiments, the coil 122 can be connected to the core 120 using gold solder, which is highly radiopaque.

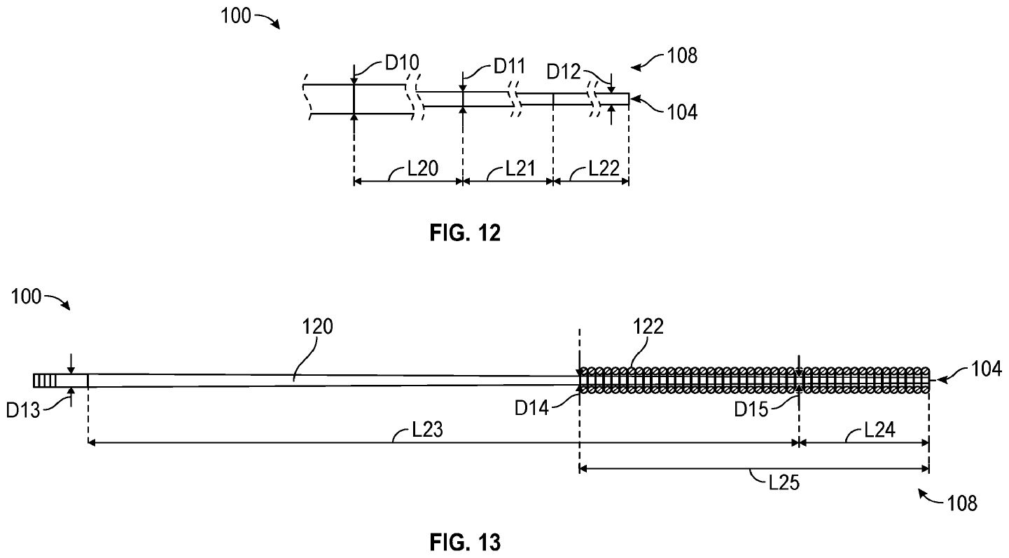

FIG. 12 shows an embodiment of a proximal region 108 of a guidewire 100. The proximal region 108 can include a coil 122 over its length extending distally, for example 300 mm or about 300 mm, from the proximal end 104, however the coil 122 is not shown for clarity. As shown, a portion of the guidewire 100 distal to the proximal region can have a diameter D10 of 0.470 mm±0.005 mm or about 0.470 mm±0.005 mm. The diameter of the guidewire 100 can taper down from the diameter D10 to a diameter D11 of 0.241 mm±0.004 mm or about 0.241 mm±0.004 mm along a length L20 as shown of 80 mm±3 mm or about 80 mm±3 mm. The diameter of the guidewire 100 can further taper down from the diameter D11 to a diameter D12 of 0.180 mm±0.004 mm or about 0.180 mm±0.004 mm along a length L21 as shown of 250 mm±3 mm or about 250 mm±3 mm. The proximal most section of the proximal region can have the diameter D12 along its length L22 as shown of 40 mm±3 mm or about 40 mm±3 mm.

FIG. 13 shows another embodiment of a proximal region 108 of a guidewire 100. The proximal region 108 can include the coil 122 as shown, which can extend distally a length L25 of 108 mm±3 mm or about 108 mm±3 mm from the proximal end 104. As shown, a portion of the guidewire 100 distal to the proximal region can have a diameter D13 of 0.470 mm±0.005 mm or about 0.470 mm±0.005 mm. The diameter of the guidewire 100 can taper down from the diameter D13 to a diameter D15 of 0.180 mm±0.004 mm or about 0.180 mm±0.004 mm along a length L23 as shown of 220 mm±3 mm or about 220 mm±3 mm. The proximal most section of the proximal region can have the diameter D15 along its length L24 as shown of 40 mm±3 mm or about 40 mm±3 mm. As further shown, the coil 122 can end distally at a portion of the guidewire 100 having a diameter D14 of 0.270 mm±0.004 mm or about 0.270 mm±0.004 mm.

In comparison to the embodiment shown in FIG. 12, the embodiment of FIG. 13 can have a decreased level of friction when passing through an MCS device and its catheter 16 due to the reduced coil length. Furthermore, the embodiment of FIG. 13 can have an increased ability to transmit longitudinal force compared to the embodiment of FIG. 12 due to a shorter length of its tapered section. Due to the shorter length of the tapered section, the embodiment of FIG. 13 may be stiffer in the proximal region 108 (e.g., to make insertion easier) than the embodiment of FIG. 12, but can have a soft proximal end to reduce friction when inserted. A proximal region 108 that lacks sufficient stiffness may result in buckling or reduced transmission of force to the proximal end 104 of the guidewire 100 when inserting the proximal end 104 into an MCS device. In certain embodiments, a diameter of the guidewire 100 greater than 0.36 mm or greater than about 0.36 mm can provide sufficient stiffness for ease of insertion of the guidewire 100 into an MCS device (for example, by preventing or reducing buckling or reduced transmission of force). In certain embodiments, a diameter smaller than 0.36 mm or smaller than about 0.36 mm, such as 0.18 mm or about 0.18 mm, at the proximal end 104 may provide a sufficiently small and soft surface for insertion of the proximal end 104 into the MCS device without significant friction. Thus, in certain embodiments, it may be beneficial to have a diameter less than 0.36 mm or less than about 0.36 mm at the proximal end 104 while having a diameter greater than 0.36 mm or greater than about 0.36 mm close enough to the proximal end 104 to prevent or reduce buckling or reduced transmission of force. The guidewire 100 of the embodiment of FIG. 13 can have a diameter greater than 0.36 mm or greater than about 0.36 mm at a length of 176 mm or about 176 mm or more from the proximal end 104 while the guidewire 100 of the embodiment of FIG. 12 can have a diameter greater than 0.36 mm or greater than about 0.36 mm at a length of 331 mm or about 331 mm from the proximal end 104. Thus, the guidewire 100 of the embodiment of FIG. 13 may provide increased stiffness sufficient to prevent or reduce buckling or reduced transmission of force at a distance closer to the proximal end 104 than the guidewire of the embodiment of FIG. 12.

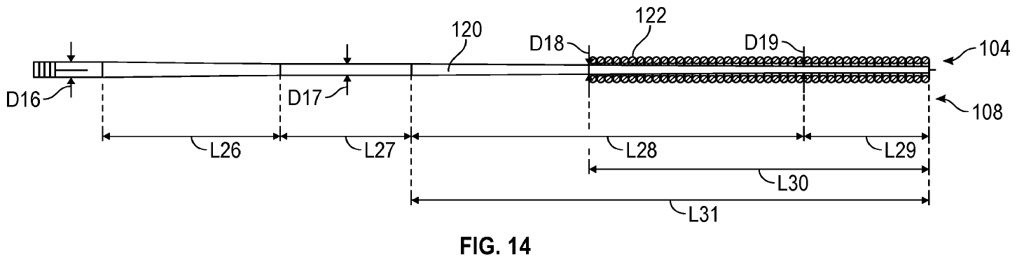

FIG. 14 shows another embodiment of a proximal region 108 of a guidewire 100. The proximal region 108 can include the coil 122 as shown, which can extend distally a length L30 of 108 mm±3 mm or about 108 mm±3 mm from the proximal end 104. As shown, a portion of the guidewire 100 distal to the proximal region can have a diameter D16 of 0.470 mm±0.005 mm or about 0.470 mm±0.005 mm. The diameter of the guidewire 100 can taper down from the diameter D16 to a diameter D17 of between 0.35 mm or about 0.35 mm to 0.40 mm or about 0.40 mm, preferably 0.360 mm±0.004 or about 0.360 mm±0.004 mm along a length L26 as shown of 20 mm or about 20 mm to 50 mm or about 50 mm. The guidewire 100 can maintain the diameter D17 along a length L27 of 1000 mm±10 mm or about 1000 mm±10 mm as shown. The diameter of the guidewire 100 can further taper down from the diameter D17 to a diameter D19 of 0.180 mm±0.004 mm or about 0.180 mm±0.004 mm along a length L28 of 220 mm±3 mm or about 220 mm±3 mm as shown. The proximal most section of the proximal region can have the diameter D19 along its length L29 as shown of 40 mm±3 mm or about 40 mm±3 mm. As further shown, the coil 122 can end distally at a portion of the guidewire 100 having a diameter D18 of 0.270 mm±0.004 mm or about 0.270 mm±0.004 mm. In use, the entire proximal region 108 of the guidewire 100 including lengths L31, L27, and L26 can pass through the full length of an MCS device and its catheter 16 before the MCS device and its catheter are inserted into the patient. Once the proximal end 104 and/or proximal region 108 exits the proximal guidewire port 37 of the proximal hub 18 of the MCS system it can be held while advancing the MCS device/catheter into the patient's vasculature, where it advances over the thicker diameter D16.

Tapered section(s) of the guidewire 100, if included, can be made by grinding the core 120 of the guidewire 100 to the desired profile. Alternatively, or in addition, such section(s) can be made as separate pieces and joined together, such as by welding, soldering, or another mechanical joint.

In some embodiments, the guidewires described herein can be used in MCS systems or mechanical left ventricular support systems, for example, for the placement of MCS devices or VADs. FIGS. 15-21 show a distal region of an embodiment of an MCS device. As shown in FIG. 15, a pump zone 60 extends between a bend relief 62 at the distal end of the catheter 16 and a distal tip 64. A pump inlet 66 is in fluid communication with a pump outlet 68 by way of a flow path extending axially through the inlet tube 70. The pump inlet 66 may be positioned at about the transition between the inlet tube 70 and the proximal end of distal tip 64, and in any event is generally within 5 cm or about 5 cm or 3 cm or about 3 cm or less from the distal port 76. The inlet tube 70 may have an axial length within the range of from about 60 mm to about 100 mm and in one implementation is 67.5 mm or about 67.5 mm. The outside diameter of the inlet tube 70 can be within the range of from 4 mm or about 4 mm to 5.4 mm or about 5.4 mm, and in one implementation is 4.66 mm or about 4.66 mm.

The impeller 72 is positioned in the flow path between the pump inlet 66 and pump outlet 68. In the illustrated embodiment, the impeller 72 is positioned adjacent to the pump outlet 68. As is discussed further below, the impeller 72 is rotationally driven by a motor contained within motor housing 74, on the proximal side of the impeller 72.

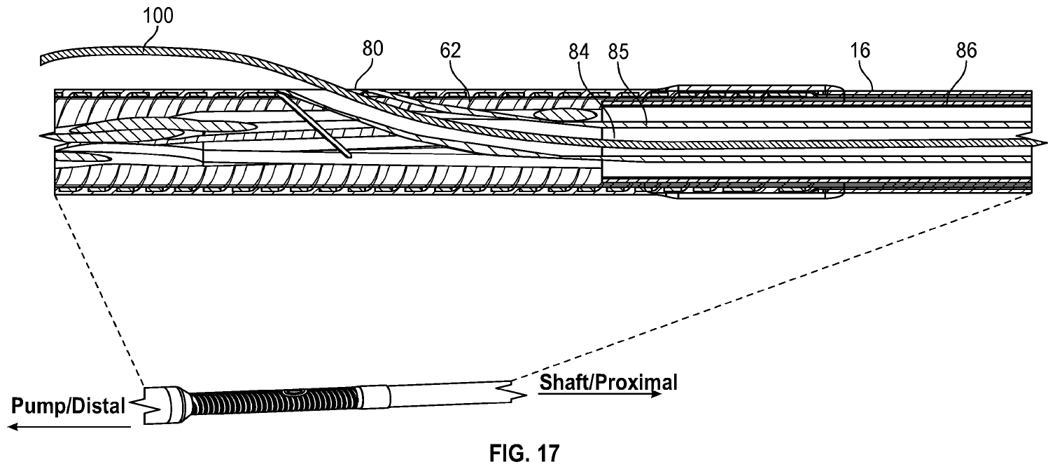

The proximal end 104 of the guidewires 100 described herein can be provided with a rounded end to facilitate entering a distal opening of a guidewire lumen 84 (shown in FIG. 17). For example, the proximal end 104 can be hemispherical, hemispheroidal, parabolic, or otherwise convex in shape. With reference to FIG. 16, the proximal end 104 and proximal region 108 can be configured to enter a distal first guidewire port 76, advance proximally to a second guidewire port 78, where the proximal end 104 exits the MCS device or VAD and extends proximally across the outside of the impeller 72 and motor housing 74 of the MCS device or VAD, and enters the catheter 16 or bend relief 62 of the MCS device or VAD via a third guidewire port 80. In some embodiments, a distance between the second guidewire port 78 and the third guidewire port 80 can be within a range of between 60 mm or about 60 mm to 95 mm or about 95 mm, within a range of between 60 mm or about 60 mm to 85 mm or about 85 mm, or within a range of between 70 mm or about 70 mm to 95 mm or about 95 mm. The third guidewire port 80 is located proximal to the motor housing 74, and, in the illustrated embodiment, is located on the bend relief 62. The third guidewire port 80 is in communication with a guidewire lumen 84 which extends proximally throughout the length of the catheter 16 and exits at the proximal guidewire port 37 of the proximal hub 18 (shown in FIG. 3).

The catheter 16 may be provided with a removable guidewire guide tube 83 which tracks the intended path of the guidewire from the first guidewire port 76, proximally through the distal tip 64 and back outside of the inlet tube 70 via second guide wire port 78 and back into the catheter 16 or bend relief 62 via third guidewire port 80. In the implementation illustrated in FIG. 16, the guidewire guide tube 83 extends proximally within the catheter 16 to a proximal end 81, in communication with, or within the guidewire lumen 84 which extends to the proximal hub 18. The guidewire guide proximal end 81 may be positioned within 5 mm or about 5 mm or 10 mm or about 10 mm of the distal end of the catheter 16, or may extend into the guidewire lumen 84 for at least 10 mm or about 10 mm or 20 mm or about 20 mm, such as within a range of from 10 mm or about 10 mm to 50 mm or about 50 mm or within a range of from 30 mm or about 30 mm to 50 mm or about 50 mm. In some embodiments, the guidewire guide tube 83 can have an inner diameter in a range of between 0.6 mm or about 0.6 mm to 1 mm or about 1 mm. The proximal end 104 of a guidewire 100 may be inserted into the first (distal) guidewire port 76 and guided along the intended path by tracking inside of the guidewire guide tube 83. The guidewire guide tube 83 may then be removed, leaving the guidewire in place.

The proximal region 108 of the guidewire 100 can thus be configured to track inside the guidewire guide tube 83 around any tight angles necessary to traverse the second guidewire port 78 and third guidewire port 80 without kinking and while retaining a low friction relationship with the guidewire lumen 84. FIG. 17 is an enlarged cross-sectional view of a portion of the bend relief 62 and catheter 16 of the MCS device showing the guidewire 100 entering the bend relief 62 via the third guidewire port 80.

The guidewire lumen 84 can be defined by a guidewire tube 85. The guidewire tube 85 can have an inner diameter of 0.8 mm or about 0.8 mm. The guidewire tube 85 can have an outer diameter of 1.1 mm or about 1.1 mm. In some embodiments, the guidewire tube 85 can have a radius of curvature in a range between 10 mm or about 10 mm and 18 mm or about 18 mm as it bends away from the central axis of the catheter 16 and toward the third guidewire port 80.

The guidewire tube 85 can be configured to minimize friction therein. In some embodiments, the guidewire tube 85 can be formed of PTFE. In some embodiments, the guidewire tube 85 can include an outer coating, such as a polyether block amide (PEBA) coating. In some embodiments, the guidewire tube 85 can be formed of polyether block amide (PEBA) and have a lubricious inner lining, such as an inner lining of PTFE, to reduce friction therein.

In some embodiments, as shown in FIG. 16, the guidewire guide tube 83 may be part of a removable guidewire aid 38. The guidewire aide 38 can include a funnel 92 to facilitate insertion of the guidewire into the guidewire port 76. In some embodiments, a distal end of the guidewire guide tube 83 is attached to a pull tab 94 of guidewire aid 38 and provided with an axially extending split line such as a weakening, slot or perforated tearable line. Removal may be accomplished such as by grasping the pull tab 94 and pulling out the guidewire guide tube 83 as it splits and peels away along the split line. In some embodiments, the inside surface of the guidewire guide tube 83 may be provided with a lubricious coating, such as PTFE.

As shown in FIG. 17, at least a portion of the guidewire tube 85 can be positioned within a tube 86 within the catheter 16. The catheter 16 can contain additional components in the space between the guidewire tube 85 and the tube 86, such as conductor wires and/or control wires for operation of the MCS device or VAD.

The guidewire tube 85 can extend distally beyond a distal end of the tube 86 to the guidewire port 80. Distal to the tube 86, additional components, such as conductor wires and/or control wires for operation of the MCS device or VAD, can be housed between the guidewire tube 85 and the inner surface of the bend relief 62.

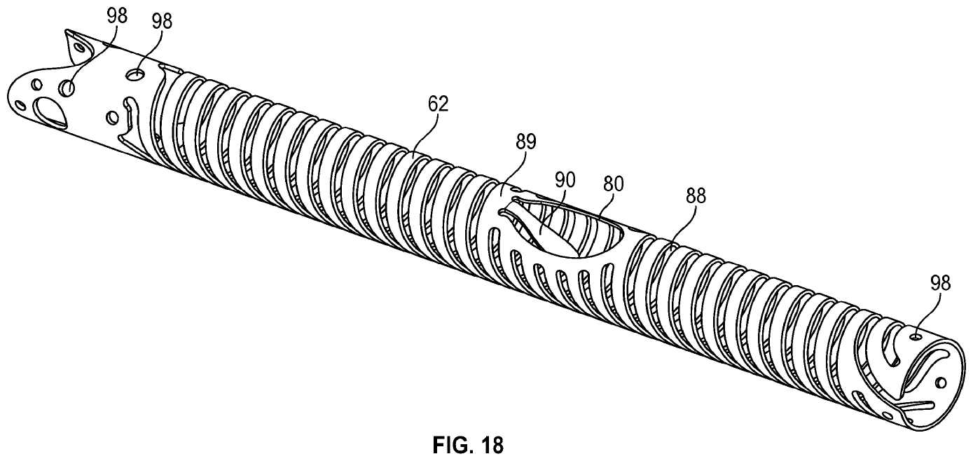

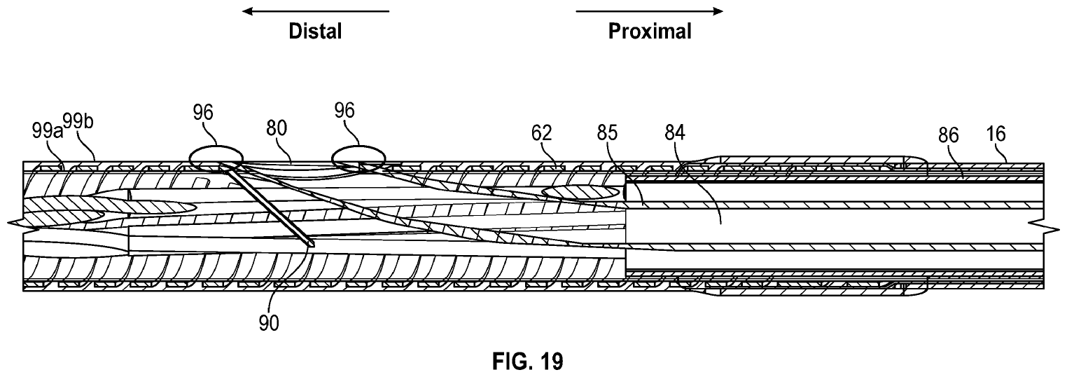

As shown in FIGS. 18-19, the bend relief 62 (also referred to as a strain relief) may be a metallic tube (e.g., stainless steel or nitinol) having a plurality of helical laser cuts 88. The bend relief 62 can be flexible, but provide increased stiffness at the transition between the motor housing 74 and the catheter 16 in comparison to the portion of the catheter 16 proximal to the bend relief 62. The stiffness of the bend relief 62 can be greater than that of the catheter 16 proximal to the bend relief 62 so as to prevent kinking while the MCS device or VAD is pushed distally, such as pushed distally over the guidewire 100.

In certain embodiments, the helical laser cuts 88 can extend along the length of the bend relief 62. A region around the third guidewire port 80 may be absent of laser cuts 88. Instead, a solid boundary 89 may extend around the third guidewire port 80.

The bend relief 62 can also include an inwardly folded flap 90. The flap 90 can extend from the boundary 89 into the inner lumen of the bend relief 62. The flap 90 of the bend relief 62 can guide the guidewire tube 85 out of the bend relief 62 and provide support to the guidewire tube 85 to maintain the guidewire tube 85 in position. In some embodiments, the guidewire tube 85 can be attached to the flap 90 by an adhesive, such as glue. The flap 90 can provide strength to the guidewire tube 85 and the third guidewire port 80. In some embodiments, the flap 90 can prevent kinking of the guidewire tube 85. In some embodiments, the flap 90 can bend inwardly toward the inner lumen of the bend relief 62 at an angle of between 30° or about 30° and 60° or about 60°, such as 45° or about 45°, from the boundary 89.

In certain embodiments, the guidewire tube 85 can be attached to the boundary 89 by an adhesive such as glue in addition to or alternatively to the flap 90. As shown in FIG. 19, in some embodiments, adhesive beads or seams 96 can be positioned over the cut edges of the boundary 89 and the guidewire tube 85. A distal end of the guidewire tube 85 can be flush with the boundary 89. The beads 96 can secure the guidewire tube 85 in position. The beads 96 can also prevent scratching between the guidewire 100 and the bend relief 62. For example, the beads 96 can be positioned so that the guidewire 100 will contact the beads 96 as the guidewire 100 moves along the guidewire path.

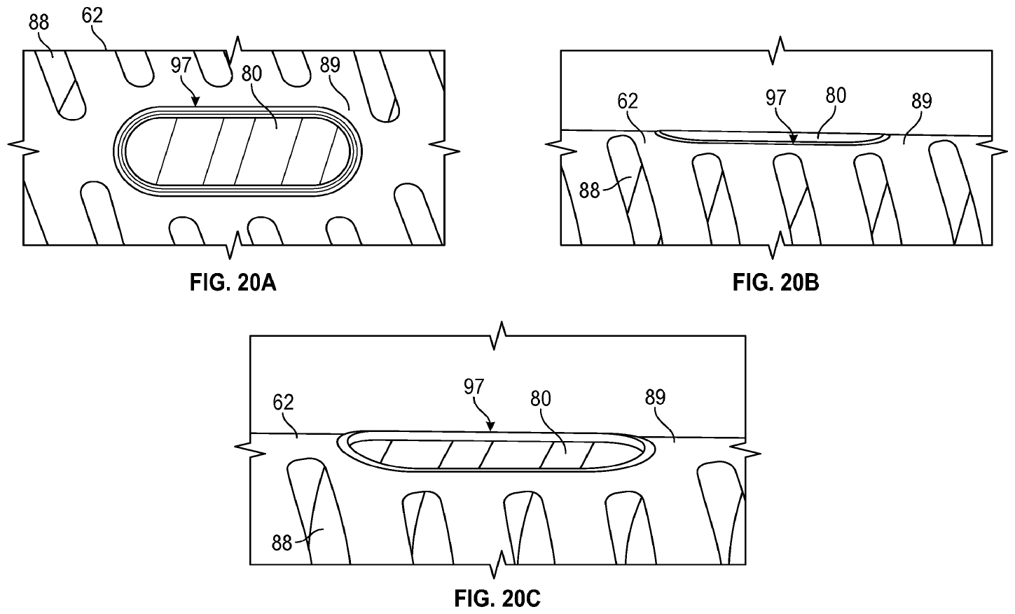

As shown in FIGS. 20A-C, in some embodiments, the bend relief 62 can include a laser weld seam 97 around the third guidewire port 80 to form a smooth edge to allow the guidewire 100 to pass through the third guidewire port 80 without catching on the boundary 89. As shown in FIGS. 20A-C, in some embodiments, the third guidewire port 80 can be elliptical, oblong, or generally elliptical or generally oblong in shape.

As shown in FIG. 19, the bend relief 62 can include an inner liner 99a positioned within the metallic tube and an outer liner 99b positioned over the metallic tube. As shown in FIG. 18, holes 98 at a proximal end and a distal end of the bend relief 62 can provide for a connection between the inner and outer liners. In some embodiments, the inner liner 99a and outer liner 99b can connect to one another through the helical laser cuts 88.

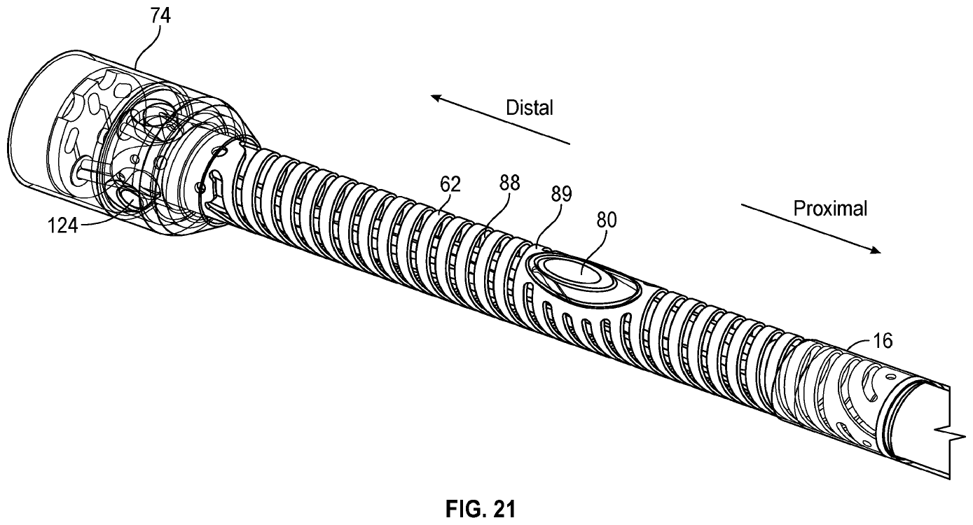

FIG. 21 is a perspective view of a portion of the distal region of the MCS device or VAD including the third guidewire port 80 and showing a junction between the motor housing 74 and the bend relief 62 and the bend relief 62 and the catheter 16. As shown in FIG. 21 the bend relief 62 can be firmly connected to the motor housing 74 with a backend pin 124 inserted through a hole in the bend relief 62 and the motor housing 74 which provides sufficient tensile strength to pull on the catheter 16 to remove or manipulate the MCS device or VAD.

In some embodiments, a method of delivering a device to a cardiovascular system of the patient can be performed using the guidewire 100. The method can include delivering an access guidewire having an outer diameter greater than a minimum outer diameter of the guidewire 100 to the cardiovascular system of the patient. For example, the access guidewire can have an outer diameter of 0.035″ or about 0.035″. After delivery of the access guidewire, the method can include advancing a catheter, such as catheter 200, over the access guidewire. As described herein, the catheter 200 can include one or more side holes. After the catheter 200 is advanced over the access guidewire, the access guidewire can be removed from the catheter 200. After the access guidewire is removed from the catheter 200, the guidewire 100 can be advanced through the catheter 200 to the cardiovascular system of the patient. As described herein, the guidewire 100 may bypass the holes of the catheter 200 without extending out of the side holes of the catheter 200 as the guidewire 100 advances through the catheter 200. After the guidewire 100 is advanced through the catheter 200, the catheter 200 can be removed from the guidewire 100 and from the patient. After the catheter 200 is removed, the proximal end 104 of the guidewire 100 can be fed into a distal end of a device or heart pump, such as an MCS device (as shown in FIGS. 15-21 for example) or a VAD. After the proximal end 104 of the guidewire 100 is fed into the distal end of the device, the device can be advanced over the guidewire 100 into the cardiovascular system of the patient (e.g., such as is shown in FIG. 1B).

In some embodiments, a method of delivering a device to a cardiovascular system of the patient can be performed using the guidewire 100 comprising one or more radiopaque markers, such as radiopaque markers 151, 152, and/or 153 described with respect to FIG. 1A. The method can include delivering the guidewire 100 through a diagnostic catheter, such as a 5 Fr catheter or the catheter 200 described herein, from an access point (e.g., femoral artery) to place the distal end of the guidewire 100 in the left ventricle. In some embodiments, the placement of the distal end of the guidewire 100 can be confirmed by visualizing and confirming the radiopaque markers 151 and/or 152 are located in the left ventricle, and the radiopaque marker 153 is located in the aorta. For example, in certain embodiments (e.g., embodiments including all three radiopaque markers 151, 152, and 153 or only radiopaque markers 151 and 153), placement of the guidewire can be confirmed by visualizing and confirming that the radiopaque marker 151 is in the left ventricle and the radiopaque marker 153 is in the aorta. In certain embodiments, the positioning of the guidewire 100 can be confirmed by visualizing and confirming that the aortic valve is positioned between the radiopaque marker 153 and one or both of the radiopaque marker 152 and the radiopaque marker 151 (for example, confirming that the aortic valve is positioned between the radiopaque marker 153 and the radiopaque marker 152). Contrast can be injected through the diagnostic catheter to verify positioning and/or visualize the heart anatomy. A fluoroscopic image can be obtained and saved. If the guidewire 100 is not positioned as desired, the guidewire 100 can be adjusted until it is positioned at the desired position within the heart anatomy. The diagnostic catheter can be removed while leaving in place the guidewire 100. In certain embodiments, without injecting more contrast, the guidewire 100 and any radiopaque markers of the guidewire (e.g., 151, 152, and/or 153) can be visualized using fluoroscopy and an assessment can be made to determine if the guidewire 100 has migrated out of the desired position (e.g., with the radiopaque markers 151 and/or 152 located in the left ventricle, and the radiopaque marker 153 located in the aorta) based at least in part on the visualization step. The guidewire 100 can be repositioned if needed (e.g., if it has migrated), and the device, such as an MCS device and/or VAD, can be delivered over the guidewire. In some embodiments, the method can include positioning (e.g., axially) a radiopaque marker of the MCS device and/or VAD relative to (e.g., adjacent to or aligned with) a radiopaque marker of the guidewire 100.

In some embodiments a method of delivering a device to a cardiovascular system of the patient can be performed using an embodiment of the guidewire 100 comprising at least radiopaque markers 151 and 153 described with respect to FIG. 1A. The method can include delivering the guidewire 100 through a diagnostic catheter, such as a 5 Fr catheter or the catheter 200 described herein, from an access point (e.g., femoral artery) to place the distal end of the guidewire 100 in the left ventricle. In some embodiments, the placement of the distal end of the guidewire 100 can be confirmed by visualizing and confirming the radiopaque marker 151 is located in the left ventricle, and the radiopaque marker 153 is located in the aorta. Contrast can be injected through the diagnostic catheter to verify positioning and/or visualize the heart anatomy. A fluoroscopic image can be obtained and saved. If the guidewire 100 is not positioned as desired, the guidewire 100 can be adjusted until it is positioned at the desired position within the heart anatomy. The diagnostic catheter can be removed while leaving in place the guidewire 100. In certain embodiments, without injecting more contrast, the guidewire 100 and any radiopaque markers of the guidewire (e.g., marker 151 and/or marker 153) can be visualized using fluoroscopy and an assessment can be made to determine if the guidewire 100 has migrated out of the desired position (e.g., with the radiopaque marker 151 located in the left ventricle, and the radiopaque marker 153 located in the aorta) based at least in part on the visualization step. The guidewire 100 can be repositioned if needed (e.g., if it has migrated), and the device, such as an MCS device and/or VAD, can be delivered over the guidewire. In some embodiments, the method can include positioning (e.g., axially) a radiopaque marker of the MCS device and/or VAD relative to (e.g., adjacent to or aligned with) a radiopaque marker of the guidewire 100.

In some embodiments, a method of delivering a device to a cardiovascular system of the patient can be performed using a guidewire 100 comprising at least radiopaque markers 152 and 153 described with respect to FIG. 1A. The method can include delivering the guidewire 100 through a diagnostic catheter. such as a 5 Fr catheter or the catheter 200 described herein, from an access point (e.g., femoral artery) to place the distal end of the guidewire 100 in the left ventricle. In some embodiments, the placement of the guidewire 100 can be confirmed by visualizing and confirming the radiopaque marker 152 is located in the left ventricle, and the radiopaque marker 153 is located in the aorta. In some embodiments, the placement of the guidewire can be confirmed by visualizing and confirming that the aortic valve is positioned between the radiopaque marker 153 and the radiopaque marker 152. In some embodiments, contrast can be injected through the diagnostic catheter to verify positioning and/or visualize the heart anatomy. A fluoroscopic image can be obtained and saved. In some embodiments, the method can include adjusting the guidewire so that the aortic valve is positioned between the marker 152 and the marker 153. The diagnostic catheter can be removed while leaving in place the guidewire 100. In certain embodiments, without injecting more contrast, the guidewire 100 and any radiopaque markers of the guidewire (e.g., marker 152 and/or marker 153) can be visualized using fluoroscopy and an assessment can be made to determine if the guidewire 100 has migrated out of the desired position (e.g., with the aortic valve positioned between the marker 152 and the marker 153) based at least in part on the visualization step. The guidewire 100 can be repositioned if needed (e.g., if it has migrated), and the device, such as an MCS device and/or VAD, can be delivered over the guidewire. In some embodiments, the method can include positioning (e.g., axially) a radiopaque marker of the MCS device and/or VAD relative to (e.g., adjacent to or aligned with) a radiopaque marker of the guidewire 100. Positioning the marker 152 and the marker 153 so that the aortic valve is between the marker 152 and the marker 153 can place the distal end of the guidewire 106 in the left ventricle and can place the pump inlet 66 within the left ventricle and the outflow section 68 within the aorta.

Any methods disclosed herein need not be performed in the order recited. The methods disclosed herein include certain actions taken by a practitioner; however, they can also include any third-party instruction of those actions, either expressly or by implication.

Conditional language used herein, such as, among others, “can,” “could,” “might,” “may,” “e.g.,” and the like, unless specifically stated otherwise, or otherwise understood within the context as used, is generally intended to convey that certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A guidewire configured to traverse a catheter having one or more side holes, the guidewire comprising:
a proximal end;
a distal end, the distal end comprising a distal advance segment configured to traverse distally through the catheter without exiting the side holes of the catheter; and
an elongate flexible body extending between the proximal end and the distal end, the elongate flexible body comprising a distal region extending between a distal transition and the distal end, the distal region comprising a spiral coil geometry.

2. The guidewire of claim 1, wherein the distal advance segment comprises an elongate straight tip.

3. The guidewire of either claim 1 or claim 2, wherein the distal advance segment comprises a length greater than a maximum diameter of the one or more side holes.

4. The guidewire of any one of the preceding claims, wherein a minimum length of the distal advance segment is between about 0.5 mm and about 3.5 mm.

5. The guidewire of any one of the preceding claims, wherein a maximum angle between a central axis of the distal advance segment and a longitudinal axis of the catheter is between about 17° and about 25°.

6. The guidewire of claim 1, wherein the distal advance segment comprises a curved advance segment extending from an inflection point at a distal end of the spiral coil geometry.

7. The guidewire of claim 6, wherein the spiral coil geometry is concave in a first direction and the distal advance segment is concave in a second direction.

8. The guidewire of claim 6, wherein the distal advance segment comprises a first curved region with a first inflection point and a second curved region with a second inflection point.

9. The guidewire of claim 8, wherein the first curved region and/or the second curved region comprises an arc length greater than a maximum diameter of the one or more side holes.

10. The guidewire of claim 9, wherein the arc length is greater than about 0.5 mm.

11. The guidewire of any one of claims 8-10, wherein a maximum angle between a longitudinal axis of the catheter and a central axis of a portion of the distal advance segment extending distally from the second inflection point is between about 5° and about 85°.

12. The guidewire of claim 11, wherein the maximum angle between the longitudinal axis of the catheter and the central axis of the portion of the distal advance segment extending distally from the second inflection point is between about 10° and about 60°.

13. The guidewire of any one of claims 8-12, wherein a maximum length of a portion of the distal advance segment extending distally from the second inflection point is between about 0.3 mm and about 4 mm.

14. The guidewire of any one of claims 8-13, wherein a radius of curvature of the first inflection point and/or the second inflection point is greater than a radius of the one or more side holes.

15. The guidewire of claim 14, wherein the radius of curvature of the first inflection point and/or the second inflection point is between about 0.5 mm and about 0.8 mm.

16. The guidewire of any one of claims 1-15, wherein a diameter at a largest cross-section of the distal advance segment is greater than or equal to a diameter of the one or more side holes.

17. The guidewire of any one of claims 1-16, wherein the distal advance segment is spheroidal in shape.

18. The guidewire of any one of claims 1-17, wherein a diameter at a largest cross-section of the distal advance segment is between about 0.8 mm and about 1 mm.

19. The guidewire of any one of claims 1-18, wherein the distal end is rounded in shape.

20. The guidewire of any one of claims 1-19, further comprising a proximal region extending between a proximal transition and the proximal end, wherein the proximal region is configured to facilitate movement of the guidewire through a non-linear path.

21. The guidewire of any one of claims 1-20, wherein the elongate flexible body comprises a core comprising a plurality of segments having different diameters.

22. The guidewire of claims 21, wherein the diameters of each of the plurality of segments of the core are between about 0.10 mm and about 0.5 mm.

23. The guidewire of any one of claims 21-22, wherein the plurality of segments having different diameters are connected to one another by one or more tapered, chamfered, conical, or frustoconical transition segments, each of the tapered, chamfered, conical, or frustoconical transition segments having a varying diameter across its length.

24. The guidewire of any one of claims 21-23, wherein the guidewire has a variable flexibility along its length.

25. The guidewire of any one of claims 21-24, wherein the elongate flexible body further comprises one or more coils of wire surrounding the core.

26. The guidewire of claims 25, wherein the one or more coils of wire surrounding the core prevent the guidewire from kinking.

27. The guidewire of any one of claims 25-26, wherein the one or more coils of wire surround at least a portion of the core of the distal region and/or the proximal region.

28. The guidewire of any one of claims 21-27, wherein the elongate flexible body further comprises a body region and/or a pump region.

29. The guidewire of claim 28, wherein the distal region and/or the proximal region have a diameter smaller than a diameter of the body region and/or the pump region.

30. The guidewire of any one of claims 28-29, wherein the one or more coils of wire surround at least a portion of the core of the body region and/or the pump region.

31. The guidewire of any one of claims 1-30, wherein the guidewire has a length of between about 2770 mm and 4030 mm.

32. The guidewire of any one of claims 1-31, wherein the distal region of the guidewire comprises one or more radiopaque markers.

33. The guidewire of any one of claims 1-31, wherein the distal region of the guidewire comprises a first radiopaque marker at or adjacent the distal end, a second radiopaque marker between about 20 mm and about 180 mm from the distal end, and/or a third radiopaque marker about 200 mm from the distal end.

34. The guidewire of any one of claims 1-31, wherein the guidewire comprises a plurality of radiopaque markers spaced evenly along at least a portion of its length to provide a scale that can be visualized by fluoroscopy.

35. The guidewire of any one of claims 1-34, wherein the proximal region of the guidewire comprises one or more visual markers.

36. A method of delivering a device to a cardiovascular system of a patient, the method comprising:
delivering a first guidewire to the cardiovascular system of the patient;
advancing a catheter over the first guidewire, the catheter comprising one or more side holes,
removing the first guidewire from the catheter;
advancing a second guidewire through the catheter, wherein the second guidewire comprises the guidewire of any one of claims 1-35, wherein the second guidewire is configured to bypass the one or more side holes of the catheter as the second guidewire advances through the catheter;
removing the catheter from the second guidewire;
feeding a proximal end of the second guidewire into a distal end of the device; and
advancing the device over the second guidewire into the cardiovascular system of the patient.

37. The method of claim 36, wherein the device comprises a heart pump.

38. The method of either claim 36 or claim 37, wherein the first guidewire has an outer diameter of about 0.035 inches.

39. The method of any one of claims 36-38, wherein the second guidewire has a minimum outer diameter of about 0.018 inches.

40. The method of any one of claims 36-39, wherein advancing the second guidewire into the cardiovascular system of the patient comprises advancing the second guidewire so that the first radiopaque marker and/or the second radiopaque marker are positioned within a left ventricle of the patient and the third radiopaque marker is positioned within an aorta of the patient.
